(19) United States
(12) Patent Application Publication
(10) Pub. No.: US 2019/0072492 A1
(43) Pub. Date: Mar. 7, 2019

(54) SYSTEMS, METHODS, AND APPARATUS FOR SPECTROSCOPIC ANALYSIS

(71) Applicant: VALISURE LLC, New Haven, CT (US)

(72) Inventor: Adam Daniel CLARK-JOSEPH, Urbana, IL (US)

(21) Appl. No.: 16/083,764

(22) PCT Filed: Mar. 10, 2017
(86) PCT No.: PCT/US2017/021881

§ 371 Date: Sep. 10, 2018

Related U.S. Application Data
(60) Provisional application No. 62/306,680, filed on Mar. 11, 2016.

(51) Int. Cl. G01N 21/65; G01J 3/44; G01N 21/27; G01N 33/15

(52) U.S. Cl. CPC G01N 21/65; G01N 33/15; G01N 21/274; G01J 3/44

(57) ABSTRACT

Disclosed herein are methods and systems for analysis of samples. In some cases, the analysis may involve spectroscopic techniques such as Raman spectroscopy. The methods and systems may be used to identify one or more characteristics of a sample, such as the identity of, and the quantity of a molecule within the sample. The methods and systems may be used to determine an impurity of the sample, or inactive excipients within the sample. In some embodiments, the methods and systems of the present disclosure provide for rapid sample analysis that is more accurate, precise and cost-effective than traditional means.

### CROSS-REFERENCE

The present application claims priority to U.S. Provisional Application No. 62/306,680 filed on Mar. 11, 2016, which is incorporated herein by reference in its entirety.

### BACKGROUND OF THE INVENTION

There is a considerable and increasing problem associated with counterfeit, impure, or substandard medications (e.g. pharmaceutical products) in both developing and developed countries. Accordingly, the ability to accurately identify and/or determine the quantity, composition or content of drugs and medicines is becoming an important issue not only for corporations and governments, but for individuals and/or intended end-users of a product. For example, a lack of oversight for verifying identity, composition, and/or quantity of ingredients in pharmaceutical products can lead to unintended and adverse consequences for individual end-users.

Various types of analysis may be used to identity and/or determine the quantity, composition or content of drugs, medicines, biologics, or other chemicals. Known techniques for identifying chemicals and verifying the integrity of pharmaceutical products may include, for example, nuclear magnetic resonance spectroscopy (NMR), mass spectroscopy (MS), high-performance liquid chromatography (HPLC), Fourier transform infrared (FT-IR) spectroscopy and Raman spectroscopy. Such techniques, however, may suffer from various shortcomings.

### SUMMARY OF THE INVENTION

The methods, devices, and systems provided herein can be used to analyze samples. In some instances, a composition of a sample may be verified or validated. For example, the existence of an ingredient of interest (e.g., active pharmaceutical ingredient, or API) may be verified and/or an amount of the ingredient of interest in the sample may be quantified and standard errors of the quantification may be determined. As another example, existence of other components of the sample (e.g., excipients, fillers, etc) may be verified and/or an amount of the other components may be quantified and standard errors of the quantification may be determined. In some examples, Raman spectroscopy may be utilized to analyze the quantity and/or composition of a sample.

Thus, in one aspect, a method of determining one or more characteristics of an active pharmaceutical ingredient (API) in a sample using spectroscopy is provided. The method comprises: a) calibrating for the API; b) processing the sample into a liquid comprising the API; c) placing the liquid comprising the API into a holder; d) generating light with aid of an optical source; e) directing, with aid of an optical arrangement, the light to the liquid; f) detecting, with aid of a detector, inelastic scattering of the light subsequent to an interaction between the light and the liquid; and g) determining, with aid of one or more processors individually or collective, the one or more characteristics of the API comprising a quantity of the API in the sample.

In some embodiments, the method comprises an initial step of receiving the sample from an individual end-user of the sample. Optionally, the method may further comprise generating a report for the individual end-user. In some embodiments, calibrating for the API enables the method to be used universally for samples of different sizes, shapes, formulations, or physical forms. Optionally, the method is performed using dispersive Raman spectroscopy. In some embodiments, the one or more characteristics of the API comprises an impurity of the ingredient. In some embodiments, the calibrating for the API step is accomplished using 20 reference standards or less. In some embodiments, the 20 reference standards comprise solutions having the API in a solvent for which a relative concentration of the API is known precisely. In some embodiments, the liquid is a liquid solution. Alternatively, the liquid is a liquid suspension. In some embodiments, the interaction between the light and the liquid uses backscatter geometry. In some embodiments, the liquid comprises a solvent. In some embodiments, the solvent is non-volatile. Optionally, the solvent comprises a vapor pressure of 40 mm Hg or less at 20° C. In some embodiments, the method further comprises filtering the sample prior to the step of placing the liquid into the holder. In some embodiments, the step of calibrating for the API comprises calibrating for the API regardless of the sample's size, shape, formulation, or physical form such that steps b) through g) can be performed for samples of different sizes, shapes, formulations, or physical forms. In some embodiments, the sample is a pharmaceutical formulation, and the step of calibrating for the API enables step b) through g) to be performed on any pharmaceutical formulation comprising the API.

In another aspect, a system for performing the method of any of the preceding claims is provided. The system comprises: a) a calibration mechanism for calibrating the system for the API; b) a processing device for processing the sample into a liquid comprising the API; c) a holder for holding the liquid comprising the API; d) an optical source configured to generate light; e) an optical arrangement configured to direct the generated light to the liquid; f) a detector configured to detect inelastic scattering of the light subsequent to an interaction between the light and the liquid; and g) one or more processors, individually or collectively, configured to determine the one or more characteristics of the API comprising a quantity of the API in the sample.

In another aspect, a method of generating a report on one or more characteristics of a sample for an individual is provided. The method comprises: a) obtaining the sample, wherein the sample comprises an active pharmaceutical ingredient (API); b) determining, with aid of one or more devices, the one or more characteristics of the sample; and c) generating, with aid of one or more processors operably coupled to the one or more devices, a report for the individual, wherein the report comprises the one or more characteristics of the sample comprising an API content of the sample, and wherein the individual is an end-user of the sample.

In some embodiments, the method further comprises preparing the sample for analysis with aid of a solvent prior to the step of determining the one or more characteristics of the sample. In some embodiments, the solvent is non-volatile. In some embodiments, the solvent comprises a vapor pressure of 40 mm Hg or less at 20° C. In some embodiments, the one or more devices comprise a backscatter Raman spectroscopy device. In some embodiments, the one or more devices are configured to determine one or more characteristics of the API for a plurality of different samples without need to calibrate for each of the plurality of different samples. In some embodiments, the plurality of different samples are of different shapes and sizes. In some embodiments, the sample is a single pill. In some embodiments, the one or more devices comprise a filter for removing one or more excipients from the sample. In some embodiments, the method further comprises a step of calibrating for the API regardless of the sample's size, shape, formulation, or physical form such that determining the one or more characteristics of the sample can be performed for samples of different sizes, shapes, formulations, or physical forms. In some embodiments, the sample is a pharmaceutical formulation, and the step of calibrating for the API enables step b) to be performed on any pharmaceutical formulation comprising the API.

In another aspect, a method of determining one or more characteristics of an active pharmaceutical ingredient (API) in a sample using spectroscopy is provided. The method comprises: a) calibrating for the API; b) processing the sample into a liquid comprising the API and a solvent; c) placing the liquid into a holder; d) generating light with aid of an optical source; e) directing, with aid of an optical arrangement, the light to the liquid; f) detecting, with aid of a detector, inelastic scattering of the light subsequent to an interaction between the light and the liquid; and g) determining, with aid of one or more processors individually or collective, a concentration of the API in the solvent.

In some embodiments, the concentration is determined through a ratiometric analysis of the API and the solvent. In some embodiments, two or more analyte concentrations are determined simultaneously through a ratiometric analysis of analytes and solvent. In some embodiments, spectral features of the API and solvent are chosen for ratiometric calibration with the aid of a database of known interfering substances, so as to minimize possible interference and optimize accuracy. In some embodiments, the API and the solvent are calibrated through a ratiometric approach utilizing 10 or less spectral features on a spectrum. In some embodiments, the API and the solvent are calibrated by utilizing 5 or less spectral features on a spectrum. In some embodiments, the API and the solvent are calibrated through a ratiometric approach utilizing 2 or more features on a spectrum. In some embodiments, the API and the solvent are calibrated through a ratiometric approach utilizing one or more combinations of spectral features. In some embodiments, the API and the solvent are calibrated through a ratiometric approach utilizing 100 or more combinations of spectral features. In some embodiments, the API and the solvent are calibrated through a ratiometric approach utilizing 1,000 or more combinations of spectral features. In some embodiments, the API and the solvent are calibrated through a ratiometric approach utilizing 10,000 or more combinations of spectral features. In some embodiments, the API and the solvent are calibrated through a ratiometric approach utilizing 100,000 or more combinations of spectral features. In some embodiments, the API and the solvent are calibrated through a ratiometric approach utilizing 1,000,000 or more combinations of spectral features. In some embodiments, API and the solvent are calibrated through a ratiometric approach utilizing 10,000,000 or more combinations of spectral features. In some embodiments, an automated optimization of analytical calibration for specific analytes is used. In some embodiments, an automated optimization of analytical calibration for specific solvents is used. In some embodiments, an automated optimization of analytical calibration for selecting calibration-relevant spectral features is used. In some embodiments, an estimation of uncertainty of the measurement is reportable for each, individual sample uniquely. In some embodiments, a non-parametric estimation of uncertainty of the measurement is reportable for each, individual sample uniquely. In some embodiments, the method is performed using dispersive Raman spectroscopy. In some embodiments, the one or more characteristics of the API comprises an impurity of the ingredient. In some embodiments, the calibrating for the API step is accomplished using 20 reference standards or less. In some embodiments, the 20 reference standards comprise solutions having the API in a solvent for which a relative concentration of the API is known precisely. In some embodiments, the method further comprises determining a quantity of the API in the sample. In some embodiments, the step of calibrating for the API comprises calibrating for the API regardless of the sample's size, shape, formulation, or physical form such that steps b) through g) can be performed for samples of different sizes, shapes, formulations, or physical forms. In some embodiments, the sample is a pharmaceutical formulation, and the step of calibrating for the API enables step b) through g) to be performed on any pharmaceutical formulation comprising the API. In some embodiments, the estimates are adjusted to correct for variation in the wavelength of the light directed to the liquid. In some embodiments, the calibration step includes calibrating for the adjustments used to correct for variation in the wavelength of the light directed to the liquid.

In another aspect, a system for performing the method of any of the preceding claims is provided. The system comprises: a) calibration mechanism for calibrating the system for the API; b) a processing device for processing the sample into a liquid comprising the API and a solvent; c) a holder for holding the liquid comprising the API and the solvent; d) an optical source configured to generate light; e) an optical arrangement configured to direct light to the liquid; f) a detector configured to detect inelastic scattering of the light subsequent to an interaction between the light and the liquid; and g) one or more processors, individually or collectively, configured to determine a concentration of the API in the solvent.

It shall be understood that different aspects of the invention can be appreciated individually, collectively, or in combination with each other. Various aspects of the invention described herein may be applied to any of the particular applications set forth below or for any other types of devices.

Other objects and features of the present invention will become apparent by a review of the specification, claims, and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a non-limiting example of a sample analysis system as described herein.

FIG. 2 depicts a non-limiting example of detection of impurities in a sample utilizing the systems described herein.

FIG. 3 depicts an industry-standard system unable to detect impurities in a sample.

FIG. 4 describes a method for determining characteristics of an ingredient in a sample as described herein.

FIG. 5 depicts a perspective view of a non-limiting mechanism for securing the holder to the device.

FIG. 6 depicts an exploded view of a non-limiting mechanism for securing the holder to the device.

FIG. 7 depicts a non-limiting example of a system as described herein.

### DETAILED DESCRIPTION OF THE INVENTION

The methods, devices, and systems of the present disclosure provide a means for determining one or more characteristics of a sample. In some instances, the characteristic may comprise identities or quantities of active ingredients (e.g. active pharmaceutical ingredient, active substances, active constituent, proteins, etc) and/or excipients. The characteristics of the sample may be determined without regards to a physical form (e.g., size, shape, etc) of the sample. For example, the methods, devices, and systems of the present disclosure may be utilized to determine characteristics of the sample without having to be calibrated differently depending on the physical form of the sample. In some instances, calibrating for an active ingredient sample may allow the methods, devices, and systems described herein to be utilized for differing samples (e.g. differing in physical forms) but comprising a same active ingredient. Optionally, processing the samples into a solution comprising the ingredient with aid of appropriate solvents may facilitate this process.

In the context of samples comprising active ingredients, counterfeit and substandard medications may be a considerable and ever growing problem, both in developing and developed economies. For example, for end-users of samples (e.g., drugs, biologics, etc), there may be no oversight in ensuring that the appropriate ingredients (e.g., both active and inactive ingredients) are incorporated in the sample, or that an appropriate amount of the active ingredient is present, oftentimes leading to improper dosing for the user. In some instances, improper excipients in the sample may lead to undesirable and adverse reactions in the user.

As described herein, samples may comprise both active ingredients, also referred to as ingredients of interest, and/or other components such as inactive ingredients or excipients. The active ingredients may refer to biologically active components in the samples. The active ingredient may confer bio-activity or pharmacological activity to the sample, and may confer therapeutic effect to the samples in some instances.

Excipients may refer to substances other than the active ingredients which are included in the manufacturing process or are contained in a finished sample dosage form. Excipients may include, but are not limited to, diluents, fillers, binders, disintegrants, lubricants, coloring agents, and preservatives. Even if inactive, the excipients may in some instances significantly affect chemical and/or physical properties of the sample and its biopharmaceutical profile. For some samples, the excipients may make up the bulk of the total dosage form

In some instances, the excipients may play an important function for the sample in various aspects. For example, the excipients may play part in the manufacturing process of the sample; the excipients may be important for keeping the active ingredient from being released too early in the assimilation process in places where it could damage tender tissue and create gastric irritation or stomach upsets; the excipients may help the sample disintegrate into particles small enough to reach the blood stream more quickly; the excipients may protect the sample's stability so it will be at maximum effectiveness at time of use; the excipients may be used to aid the identification of the sample; the excipients may be used simply to make the sample taste and look better.

The methods, devices, and systems provided herein allow for the analysis of small quantities of samples such as pills, tablets, capsules, etc. For example, the present disclosure provides the means for analyzing individual samples from received from an end user. Optionally, the samples may be received from the end user on a regular and/or semi-regular basis and a report comprising results of the analysis may be sent to the client (e.g. the end user). Accordingly, the present disclosure provides a means for individuals to get analysis done for samples (e.g., drugs, medications, biologics, etc) they may be taking. The analysis may provide information regarding both active ingredients and/or inactive ingredients. For example, the analysis may help determine an identity of the active ingredients and/or the inactive ingredients. In some instances, the analysis may help quantify an amount of the active ingredients and/or the inactive ingredients.

Advantageously, certain properties of the methods, devices, and systems (e.g., ability to provide analysis for individual samples or pills, ability to provide analysis to samples of differing physical sizes or shapes without having to recalibrate for the differing sizes and shapes, etc) may enable the servicing of individual end-users of samples for the analysis of the samples (e.g., batch of samples).

For example, an individual may receive a batch of prescription drugs to be taken but may be uncertain that it contains the appropriate active ingredients, or an appropriate amount of the active ingredient. In some instances, the individual may be cautious of whether the drug contains inappropriate excipients. The individual may send in one or more drugs of the batch, and the disclosure provided herein may provide a means for the determining the characteristics of the drug, e.g., identities and/or quantities of the active ingredients and/or excipients. After receiving a report, or result of the analysis, the individual may be reassured that the sample is as expected, or informed that the sample is substandard.

In certain aspects, methods, devices, and systems are provided for the analysis of a sample. In some cases, the methods, devices, and systems can be used to determine one or more characteristics of a sample, including an identity, composition, or quantity of components within the sample. The components may include active ingredients and/or excipients. The methods, devices, and systems may overcome the various shortcomings experienced by existing technologies. For example, the systems may have low instrument cost, utilize consumables with low cost, provide quick and accurate analysis, be capable of accomplishing analyses of mixtures, be capable of analyzing low concentrations, be capable of analyzing or identifying a unique chemical ID, be capable of identifying solids, and not require expertise or special technical training for operation. In addition, the systems may be capable of analyzing a plurality of different samples having differing sizes, shapes, density, etc without having a need for separate calibration. In some instances, the systems may be capable of analyzing a plurality of different samples if the samples have a same ingredient of interest, e.g. active pharmaceutical ingredient. In some instances, the system may be capable of universally analyzing samples having a same ingredient of interest, e.g. without calibrations specific to the different samples. In some instances, the system may be capable of universally analyzing samples having a same ingredient of interest, e.g. without calibration in between the different samples.

The methods, devices, and systems can be used to analyze a sample provided by an end-user. For example, a composition of a sample may be verified or validated. As another example, an amount of an ingredient of interest in the sample may be quantified for the end-user. In some examples, Raman spectroscopy may be utilized to analyze the quantity or composition of a sample (e.g., a pill, tablet, capsule, etc). In some cases, the methods, devices, and systems may provide improvements to existing technologies. For example, the time from sample to answer may be substantially faster than traditional methods. Optionally, the methods, devices, and systems may provide more accurate quantifications than traditional methods. In some cases, the methods may be cheaper to perform than traditional methods and may not require expertise or special technical training for operation.

General

The present disclosure provides methods, devices, and systems for analyzing samples having varying features and obtaining their characteristics. For examples, the samples may vary in a size, chemical composition (e.g. excipient formulation and/or API dosage), density, shape, etc. The methods, devices, and systems may be universally applicable to the samples of varying features and may be capable of obtaining characteristics for each, e.g. without calibrating for the varying features. In some instances, the methods, devices, and systems may be universally applicable to the samples of varying features having a same active ingredient and may be capable of obtaining characteristics for each with only having to calibrate for the active ingredient. While the disclosure is described herein primarily with regard to use in the context of Raman Spectroscopy, and dispersive Raman Spectroscopy, it could readily be utilized with other spectroscopic techniques or analysis techniques.

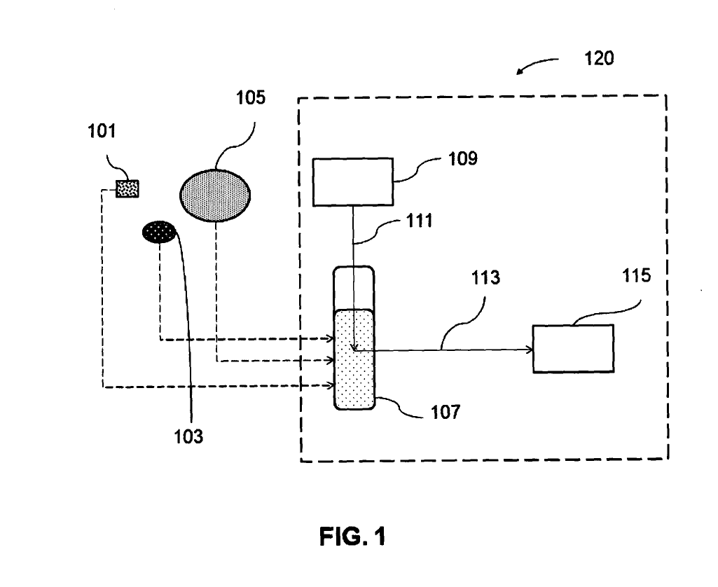

FIG. 1 schematically illustrates a non-limiting example of a sample analysis system in accordance with aspects of the invention. One or more samples 101, 103, 105 are received by an operator of the system. The sample may be received or obtained from an end-user, or multiple end-users who are desirous of confirming or validating the contents of the sample. For example, an end-user may obtain a pill from a pharmacy. As another example, the end-user may purchase an over the counter pill from a convenience store. As used herein, end-user may refer to an actual end-user who purchases the sample or obtains the sample through any legal means. Optionally, the end-user may also refer to an entity who obtains the sample through any legal means after the sample has left its origin of manufacture in its intended form.

The sample may contain an ingredient of interest. In some instances, the sample may contain two or more ingredients of interest. The ingredient of interest may be an active ingredient, such as a pharmaceutically active ingredient. Optionally, the sample may further contain any number of additional ingredients, such as excipients. Different samples may comprise different excipients. Alternatively, different samples may comprise the same excipients. The sample can be of any composition and of any shape or size. For example, the sample may be large as provided by sample 105 or may be small as provided by sample 103. In some instances, the sample may be circular or oval as provided by samples 103, 105 or may be rectangular as provided by sample 101. The sample may be in any given form, e.g. solid, liquid, gel, cream, or any other physical form. The sample may be a prescription or over-the-counter drug or medicine as described herein.

The contents of the sample may be analyzed. In some instances, the contents of the sample may be analyzed using an analysis system 120. The analysis system may comprise a single integrated device. Alternatively, the analysis system may comprise a plurality of different devices. In some instances, the analysis system may comprise a Raman Spectrometer and the sample may be analyzed using Raman Spectroscopy, e.g. dispersive Raman Spectroscopy. Optionally, all samples comprising a same ingredient of interest (e.g. same API) may be universally analyzed using the methods and systems provided herein, e.g. without need for calibration steps between the analysis.

The sample may be in a solid, liquid, gelatinous, cream, and/or powder form. In some instances, the sample may be contained in a coating. In some aspects, the sample may be processed, e.g. prior to analysis. For example, if the sample is in a dry composition, the sample may be crushed prior to sample analysis, such as into a powder or fine particulate matter. Optionally, the samples, whether dry, wet, or in some other form, may be further dissolved or diluted into a solvent, substantially as described below. The solution may comprise the ingredient of interest of the sample. In some instances, the ingredient of interest may be evenly distributed within the solution such that any portion of the solution may be analyzed to obtain useful information regarding the sample.

The resulting solution may be placed into a holder 107 such as a vial or a vessel. In some instances, each of the different samples 101, 103, 105 may be processed prior to analysis. For example, each of the different samples 101, 103, 105 may be crushed, and/or dissolved in a solvent and placed into a holder. The processing step may enable the systems and methods provided herein to be applicable universally for a given ingredient of interest. For example, the processing step may normalize the different samples 101, 103, 105 that have varying features (e.g. varying shapes, sizes, density, excipients, etc) such that they can be analyzed by the systems and methods provided herein without need for intermediary calibration steps. For example, sample 101 may be processed and analyzed using analysis system 120. Sequentially, sample 103 may be processed and analyzed using the analysis system. Sequentially, sample 105 may be processed and analyzed using the analysis system. A single calibration step may be performed for the analysis system prior to the sequential analysis. The calibration step may calibrate for the ingredient of interest common to sample 101, 103, and 105. Subsequently, the analysis system may be applicable to be used for analyzing any sample (e.g. solution comprising the sample) comprising the ingredient of interest without further calibration steps.

Optionally, the holder may be secured in place (e.g. within the analysis system) by a clamp. The clamp may be of any configuration that secures the holder to the device. The clamp may ensure that the holder is substantially in a same position for implementing analysis of the sample and may eliminate background fluorescence from affecting a result that is obtained.

The holder comprising the sample (e.g. processed sample) may be interrogated by a spectrometer, such as a Raman Spectrometer. The Raman Spectrometer may be a back-scattering Raman spectrometer or a transmission Raman spectrometer. In some instances, a light source 109, such as a laser, is provided and directs a light 111 to the holder comprising the sample. The light, in some instances, may be a monochromatic light. The photons of the laser light are absorbed by the sample and then reemitted 113. The frequency of the reemitted photons may be shifted up or down. Upon the inelastic scattering of the light upon interaction with the sample, the reemitted photons may be collected by a detector 115. The detector may detect a shift in frequency of the reemitted photons. This shift in frequency that is detected may provide information regarding characteristics of the sample.

The characteristics of the sample may comprise, but are not limited to, an amount of the ingredient of interest (e.g. API content of the sample), a chemical ID of the sample, existence of an impurity of the sample, or identity of the sample. In some instances, the amount, or quantity of the ingredient (e.g. API content) may further be characterized. Accordingly, the characteristics of the sample may comprise a standard error for the ingredient of interest, or bootstrap confidence interval for the amount of the ingredient. For example, an uncertainty, or standard error for the amount may be determined. In some instances, a bootstrap distribution for the amount of the ingredient may be generated for the sample and/or an estimation of uncertainty for individual samples may be determined.

The analysis system 120 may provide accurate measurement of a content (e.g. quantity) of the ingredient of interest. In some instances, the analysis system may provide information regarding characteristics of the sample in a short period of time, substantially as described below. In addition, by being universally applicable to samples having varying features (e.g. if they have a same ingredient of interest), the methods and systems provided herein may enable individualized sample analysis for users, e.g. end-users. Optionally, the information regarding the characteristics of the sample may be provided on a report to an end-user of the sample.

Methods

In some aspects, a method is provided for determining characteristics of an ingredient in a sample. The method comprises: a) calibrating, with aid of a calibration means, for the ingredient; b) placing a liquid comprising the ingredient in a holder; c) generating light with aid of an optical source; d) directing, with aid of an optical arrangement, the light to the liquid; e) detecting, with aid of a detector, properties of the light subsequent to the interaction; and f) determining, with aid of one or more processors individually or collective, the characteristics of the ingredient. Optionally, the sample may be processed into a liquid form, e.g. to make the liquid comprising the ingredient that is placed in the holder.

In some aspects, a method is provided for determining characteristics of a sample comprising an active pharmaceutical ingredient (API). The method comprises: a) placing a solution in a holder, wherein the solution comprises the sample and a solvent; b) generating light with aid of an optical source; c) directing, with aid of an optical arrangement, the light to the sample; d) detecting, with aid of a detector, properties of the light subsequent to the interaction; and e) determining, with aid of one or more processors individually or collectively, the characteristics of the sample, wherein the characteristics comprise an API content of the sample, and wherein the API content is determined with less than 3% error. Optionally, the sample may be processed into a liquid form, e.g. to make the liquid comprising the ingredient that is placed in the holder.

In some aspects, a method is provided for determining characteristics of a sample comprising an active pharmaceutical ingredient (API). The method comprises: a) placing a solution in a holder, wherein the solution comprises the sample and a solvent; b) generating light with aid of an optical source; c) directing, with aid of an optical arrangement, the light to the sample; d) detecting, with aid of a detector, properties of the light subsequent to the interaction; and e) determining, with aid of one or more processors individually or collectively, the characteristics of the sample, wherein the characteristics comprise an API content of the sample, and wherein steps b) through e) takes place within 30 minutes or less. Optionally, the sample may be processed into a liquid form, e.g. to make the liquid comprising the ingredient that is placed in the holder.

In some aspects, methods are provided for generating a report on characteristics of a sample for an individual. The method comprises: a) receiving the sample from the individual, wherein the sample comprises an active pharmaceutical ingredient (API); b) determining, with aid of a device, characteristics of the sample; and c) generating, with aid of one or more processors operably coupled to the device, a report for the individual, wherein the report comprises the characteristics of the sample, and wherein the individual is an end-user of the sample. Optionally, the sample may be processed into a liquid form, e.g. to make the liquid comprising the ingredient that is placed in the holder.

In certain aspects, a sample containing an ingredient is obtained. The sample can be provided by an individual. The individual may be, for example, an end-user of a product from which the sample is obtained. The end-user may refer to a potential user of the product. In some instances, the end-user may be an intended user of the product. In some instances, the end-user may refer to a purchaser of the product. For example, the end-user may be a patient who gets prescribed prescription drugs. For example, the end-user may be a consumer who purchases an over the-counter-drug. The term “product” as used herein may refer, in some cases, to a source of the sample (e.g., the sample is a portion of the product or is obtained from the product), or the terms “sample” and “product” may be used interchangeably and may refer to the same entity. The sample may be in a liquid state, a solid state, or a semi-solid state, such as a gel, cream, or paste. In some instances, the sample is a drug. Alternatively or in addition, the sample is a biologic, biopharmaceutical, supplement, or veterinary drug. In some cases, the sample is a pharmaceutical sample. In some cases, the pharmaceutical sample is in a solid form, non-limiting examples including, pills, tablets, capsules, powders and the like. In some cases, the pharmaceutical sample is a drug prescribed by a licensed healthcare practitioner. In other cases, the pharmaceutical sample is a drug purchased over-the-counter, for example, at a drug store. In some cases, the pharmaceutical sample is a drug purchased e.g. over the Internet. In some cases, an end-user of a product from which the pharmaceutical sample is obtained is desirous of verifying or confirming the composition of the product.

In some cases, the sample contains one or more ingredients. Ingredients may be active ingredients or inactive ingredients. In some instances, the active ingredients may be biologically or chemically active ingredients. In some cases, the sample is a pharmaceutical sample and contains one or more active pharmaceutical ingredients (API). The terms “active pharmaceutical ingredients” or “API” may refer to an ingredient that is biologically active. In some cases, the pharmaceutical sample contains one API. In some cases, the pharmaceutical sample contains more than one API. In some aspects, the methods, devices, and systems provided herein are utilized to determine one or more characteristics of one or more APIs contained within a sample. Any API can be interrogated utilizing the methods, devices, and systems provided herein. Non-limiting examples of APIs suitable for interrogation with the methods, devices, and systems described herein may include; Hydrocodone/APAP (Brand Name: Vicodin®); Amoxicillin (Brand Name: Amoxil®); Lisinopril (Brand Name: Prinivil®); Esomeprazole (Brand Name: Nexium®); Atorvastatin (Brand Name: Lipitor®); Simvastatin (Brand Name: Zocor®); Clopidogrel (Brand Name: Plavix®); Montelukast (Brand Name: Singulair®); Rosuvastatin (Brand Name: Crestor®); Metoprolol (Brand Name: Lopressor®); Escitalopram (Brand Name: Lexapro®); Azithromycin (Brand Name: Zithromax®); Albuterol (Brand Name: ProAir® HFA); Hydrochlorothiazide (Brand Name: HCTZ); Metformin (Brand Name: Glucophage®); Sertraline (Brand Name: Zoloft®); Ibuprofen (Brand Name: Advil®); Zolpidem (Brand Name: Ambien®); Furosemide (Brand Name: Lasix®); Omeprazole (Brand Name: Prilosec®); Trazodone (Brand Name: Desyrel®); Valsartan (Brand Name: Diovan®); Tramadol (Ultram®); Duloxetine (Brand Name: Cymbalta®); Warfarin (Brand Name: Coumadin®); Amlodipine (Brand Name: Norvasc®); Oxycodone/APAP (Brand Name: Percocet®); Quetiapine (Brand Name: Seroquel®); Promethazine (Brand Name: Phenergan®); Fluticasone (Brand Name: Flonase®); Alprazolam (Brand Name: Xanax®); Clonazepam (Brand Name: Klonopin®); Benazepril (Brand Name: Lotensin®); Meloxicam (Brand Name: Mobic®); Citalopram (Brand Name: Celexa®); Cephalexin (Brand Name: Keflex®); Tiotropium (Brand Name: Spiriva®); Gabapentin (Brand Name: Neurontin®); Aripiprazole (Brand Name: Abilify®); Cyclobenzaprine (Brand Name: Flexeril®); Methylprednisolone (Brand Name: Medrol®); Methylphenidate (Brand Name: Ritalin®); Fexofenadine (Brand Name: Allegra®); Carvedilol (Brand Name: Coreg®); Carisoprodol (Brand Name: Soma®); Digoxin (Brand Name: Lanoxin®); Memantine (Brand Name: Namenda®); Atenolol (Brand Name: Tenormin®); Diazepam (Brand Name: Valium®); Oxycodone (Brand Name: OxyContin®); Risedronate (Brand Name: Actonel®); Folic Acid (Brand Name: Folvite®); Olmesartan (Brand Name: Benicar®); Prednisone (Brand Name: Deltasone®); Doxycycline (Brand Name: Vibramycin®); Alendronate (Brand Name: Fosamax®); Pantoprazole (Brand Name: Protonix®); Tamsulosin (Brand Name: Flomax®); Triamterene/HCTZ (Brand Name: Dyazide®); Paroxetine (Brand Name: Paxil®); Buprenorphine (Brand Name: Suboxone®); Enalapril (Brand Name: Vasotec®); Lovastatin (Brand Name: Mevacor®); Pioglitazone (Brand Name: Actos®); Pravastatin (Brand Name: Pravachol®); Fluoxetine (Brand Name: Prozac®); Insulin Detemir (Brand Name: Levemir®); Fluconazole (Brand Name: Diflucan®); Levofloxacin (Brand Name: Levaquin®); Rivaroxaban (Brand Name: Xarelto®); Celecoxib (Brand Name: Celebrex®); Codeine/APAP (Brand Name: Tylenol® #2); Mometasone (Brand Name: Nasonex®); Ciprofloxacin (Brand Name: Cipro®); Insulin Aspart (Novolog®); Venlafaxine (Brand Name: Effexor®); Lorazepam (Brand Name: Ativan®); Ezetimibe (Brand Name: Zetia®); Estrogen (Brand Name: Premarin®); Allopurinol (Brand Name: Zyloprim®); Penicillin (Brand Name: Pen VK®); Sitagliptin (Brand Name: Januvia®); Amitriptyline (Brand Name: Elavil®); Clonidine (Brand Name: Catapres®); Latanoprost (Brand Name: Xalatan®); Lisdexamfetamine (Brand Name: Vyvanse®); Niacin (Brand Name: Niaspan®); Naproxen (Brand Name: Aleve®); Dexlansoprazole (Brand Name: Dexilant®); Glyburide (Brand Name: Diabeta®); Olanzapine (Brand Name: Zyprexa®); Tolterodine (Brand Name: Detrol®); Ranitidine (Brand Name: Zantac®); Famotidine (Brand Name: Pepcid®); Diltiazem (Brand Name: Cardizem®); Insulin Glargine (Brand Name: Lantus®); Thyroid (Brand Name: Armour Thyroid®); Bupropion (Brand Name: Wellbutrin®); Cetirizine (Zyrtec®); Topiramate (Brand Name: Topamax®); Valacyclovir (Brand Name: Valtrex®); Eszopiclone (Brand Name: Lunesta®); Acyclovir (Brand Name: Zovirax®); Cefdinir (Brand Name: Omnicef®); Clindamycin (Brand Name: Cleocin®); Colchicine (Brand Name: Colcrys®); Gemfibrozil (Brand Name: Lopid®); Guaifenesin (Brand Name: Robitussin®); Glipizide (Brand Name: Glucotrol®); Irbesartan (Brand Name: Avapro®); Metoclopramide (Brand Name: Reglan®); Losartan (Brand Name: Cozaar®); Meclizine (Brand Name: Dramamine®); Metronidazole (Brand Name: Flagyl®); Vitamin D (Brand Name: Caltrate®); Testosterone (Brand Name: AndroGel®); Ropinirole (Brand Name: Requip®); Olopatadine (Brand Name: Patanol®); Moxifloxacin (Brand Name: Avelox®); Enoxaparin (Brand Name: Lovenox®); Fentanyl (Brand Name: Duragesic®); Dicyclomine (Brand Name: Bentyl®); Bisoprolol (Brand Name: Zebeta®); Atomoxetine (Brand Name: Strattera®); Ramipril (Brand Name: Altace®); Temazepam (Brand Name: Restoril®), Phentermine (Brand Name: Adipex® P); Quinapril (Brand Name: Accupril®); Sildenafil (Brand Name: Viagra®); Ondansetron (Brand Name: Zofran®); Oseltamivir (Brand Name: Tamiflu®); Methotrexate (Brand Name: Rheumatrex®); Dabigatran (Brand Name: Pradaxa®); Budesonide (Brand Name: Uceris®); Doxazosin (Brand Name: Cardura®); Desvenlafaxine (Brand Name: Pristiq®); Insulin Lispro (Brand Name: Humalog®); Clarithromycin (Brand Name: Biaxin®); Buspirone (Brand Name: Buspar®); Finasteride (Brand Name: Proscar®); Ketoconazole (Brand Name: Nizoral®); Solifenacin (Brand Name: VESlcare®); Methadone (Brand Name: Dolophine®); Minocycline (Brand Name: Minocin®); Phenazopyridine (Brand Name: Pyridium®); Spironolactone (Brand Name: Aldactone®); Vardenafil (Brand Name: Levitra®); Clobetasol (Brand Name: Clovate®); Benzonatate (Brand Name: Tessalon®); Divalproex (Brand Name: Depakote®); Dutasteride (Brand Name: Avodart®); Febuxostat (Brand Name: Uloric®); Lamotrigine (Brand Name: Lamictal®); Nortriptyline (Brand Name: Pamelor®); Roflumilast (Brand Name: Daliresp®); Rabeprazole (Brand Name: Aciphex®); Etanercept (Brand Name: Enbrel®); Nebivolol (Brand Name: Bystolic®); Nabumetone (Brand Name: Relafen®); Nifedipine (Brand Name: Procardia®); Nitrofurantoin (Brand Name: Macrobid®); Nitroglycerine (Brand Name: NitroStat® SL); Oxybutynin (Brand Name: Ditropan®); Tadalifil (Brand Name: Cialis®); Triamcinolone (Brand Name: Kenalog®); Rivastigmine (Brand Name: Exelon®); Lansoprazole (Brand Name: Prevacid®); Cefuroxime (Brand Name: Ceftin®); Methocarbamol (Brand Name: Robaxin®); Travoprost (Brand Name: Travatan®); Lurasidone (Brand Name: Latuda®); Terazosin (Brand Name: Hytrin®); Sumatriptan (Brand Name: Imitrex®); Raloxifene (Brand Name: Evista®); Mirtazepine (Brand Name: Remeron®); Adalimumab (Brand Name: Humira®); Benztropine (Brand Name: Cogentin®); Baclofen (Brand Name: Gablofen®); Hydralazine (Brand Name: Apresoline®); Mupirocin (Brand Name: Bactroban®); Propranolol (Brand Name: Inderal®); Varenicline (Brand Name: Chantix®); Verapamil (Brand Name: Verelan®); Clotrimazole (Brand Name: Lotrimin®); Phenytoin (Brand Name: Dilantin®); Pramipexole (Brand Name: Mirapex®); Liraglutide (Brand Name: Victoza®); Ticagrelor (Brand Name: Brilinta®); Diclofenac (Brand Name: Voltaren®); Saxagliptin (Brand Name: Onglyza®); Lomitapide (Brand Name: Juxtapid®); Tizanidine (Brand Name: Zanaflex®); Amphetamine /Dextro-amphetamine (Brand Name: Adderall®); Zoster Vaccine (Brand Name: Zostavax®); Ezetimibe/Simvastatin (Brand Name: Vytorin®); Vilazodone (Brand Name: Vybriid®); Hydroxyzine (Brand Name: Vistaril®); Donepezil (Brand Name: Aricept®); Acetaminophen (Brand Name: Tylenol®); and Oxcarbazepine (Brand Name: Trileptal®).

In some aspects, the methods and systems described herein may be suitable to analyze samples containing ingredients with a molecular weight. In some cases, the molecular weight of the ingredient is 100 Daltons (Da) or less. In some cases, the molecular weight of the ingredient is equal to or less than about 150 kDa, 100 kDa, 75 kDa, 50 kDa, 25 kDa, 10 kDa, 5000 Da, 4000 Da, 3000 Da, 2000 Da, 2500 Da, 1500 Da, 1000 Da, 950 Da, 900 Da, 850 Da, 800 Da, 750 Da, 700 Da, 650 Da, 600 Da, 550 Da, 500 Da, 450 Da, 400 Da, 350 Da, 300 Da, 250 Da, 200 Da, 150 Da, 100 Da, 95 Da, 90 Da, 85 Da, 80 Da, 75 Da, 70 Da, 65 Da, 60 Da, 55 Da, 50 Da, 45 Da, 40 Da, 35 Da, 30 Da, 25 Da, 20 Da, 15 Da, 10 Da, 5 Da. In some cases, the active ingredient is a protein or peptide. In some cases, the active ingredient is a small molecule or a small molecular compound.

In some cases, the pharmaceutical sample may contain one or more excipients. An excipient may be an inactive ingredient that is biologically inert. In some cases, the methods, devices, and systems described herein are capable of distinguishing between the ingredient (e.g. an API) and the one or more excipients. In some cases, the one or more excipients are filtered or otherwise removed from the sample prior to analysis. In some cases, the methods provide for filtering at least a portion of the one or more excipients from the sample prior to analysis. Excipients may include, for example, emulsifiers, stabilizers, suspending agents, binders, viscosity-increasing agents, disintegrants, antiseptics, antimicrobial agents, preservatives, disinfectants, solvents, antioxidants, diluents, sugar coatings, sweeteners, adsorbents, anticaking agents, glidants, emulsion stabilizers, thermal stabilizers, water-absorbing agents, lubricants, chelators, film-formers, granulating agents, extended release agents, stiffening agents, cationic surfactants, non-ionic surfactants, anionic surfactants, detergents, wetting agents, reducing agents, buffering agents, nutrients, dietary supplements, clouding agents, anti-foaming agents, emollients, colorants, coating agents, flavoring fixatives, fillers, gelling agents, humectants, plasticizers, tonicity agents, stabilizing agents, thickening agents, rate-controlling polymers, lyophilization aids, bulking agents, dissolution aids, ointment bases, suppository bases, water-miscible cosolvents, mucoadhesives, dispersing agents, coemulsifying agents, alkalizing agents, acidifying agents, skin penetrants, carbonating agents, sequestering agents, opacifiers, and pigments. Non-limiting examples of excipients may include acacia, alginate, alginic acid, aluminum acetate, benzyl alcohol, butyl paraben, butylated hydroxy toluene, citric acid, calcium carbonate, candelilla wax, croscarmellose sodium, confectioner sugar, colloidal silicone dioxide, cellulose, plain or anhydrous calcium phosphate, carnuba wax, corn starch, carboxymethylcellulose calcium, calcium stearate, calcium disodium EDTA, copolyvidone, castor oil hydrogenated, calcium hydrogen phosphate dehydrate, cetylpyridine chloride, cysteine HCL, crosspovidone, dibasic calcium phosphate, disodium hydrogen phosphate, dimethicone, erythrosine sodium, ethyl cellulose, gelatin, glyceryl monooleate, glycerin, glycine, glyceryl monostearate, glyceryl behenate, hydroxy propyl cellulose, hydroxyl propyl methyl cellulose, hypromellose, HPMC phthalate, iron oxides or ferric oxide, iron oxide yellow, iron oxide red or ferric oxide, lactose hydrous or anhydrous or monohydrate or spray dried, magnesium stearate, microcrystalline cellulose, mannitol, methyl cellulose, magnesium carbonate, mineral oil, methacrylic acid copolymer, magnesium oxide, methyl paraben, povidone or polyvinylpyrrolidone (PVP), polyethylene glycol (PEG), polysorbate 80, propylene glycol, polyethylene oxide, propylene paraben, polaxamer 407 or 188 or plain, potassium bicarbonate, potassium sorbate, potato starch, phosphoric acid, polyoxyl40 stearate, sodium starch glycolate, starch pregelatinized, sodium crossmellose, sodium lauryl sulfate, starch, silicon dioxide, sodium benzoate, stearic acid, sucrose, sorbic acid, sodium carbonate, saccharin sodium, sodium alginate, silica gel, sorbiton monooleate, sodium stearyl fumarate, sodium chloride, sodium metabisulfite, sodium citrate dehydrate, sodium starch, sodium carboxy methyl cellulose, succinic acid, sodium propionate, titanium dioxide, talc, triacetin, and triethyl citrate.

The pharmaceutical sample may additionally contain one or more impurities. An impurity may be, for example, formed during the manufacturing process such as unreacted starting material or intermediates or byproducts. Impurities may include degradation products such as those formed during the synthetic process, during storage, during formulation of the dosage form or during aging of the drug. Additional impurities may include inorganic impurities, stereoisomeric impurities, structural isomer impurities, reagents, ligands, catalysts, heavy metals, filter aids, charcoal, and residual solvents. In some cases, the impurities are formulation-related impurities such as method related, environmental related (e.g., exposure to adverse temperatures, light (e.g., U.V.), or humidity), and dosage form related such as mutual interaction amongst ingredients, and functional group related degradation (e.g., ester hydrolysis, hydrolysis, oxidative degradation, photolytic cleavage, or decarboxylation). In some cases, the methods, devices, and systems described herein are capable of distinguishing between an API and one or more impurities contained in the sample.

In some instances, the sample is a cosmetic. Cosmetics may include, for example, a cream or a gel. In other instances, the sample is a food, beverage, or a nutritional supplement. In some instances, the sample comprises herbicides, pesticides, or fertilizers. In some instances, the sample comprises chemical precursors used in the synthesis of other substances or materials. In some instances, the sample is a sample suspected of containing illicit drugs. In some instances, the sample comprises stimulating drugs, depressant drugs, opioids, or hallucinogenic drugs. As non-limiting samples, the sample may comprise cathinone, GHB, heroin, 1-butyl-3-(1-naphothoyl)indole (JWH-073), psilocybin, or LSD. In some instances, the sample comprises depressants or sedatives. In some instances, the sample is a substance being investigated as a possible pharmaceutical. For example, the sample may be a research sample that is still in a research and development stage. In some instances, the sample may be a sample (e.g. drug sample) in a pre-clinical or clinical-trial stage.

In some aspects, a sample is obtained. The sample may be received from an end-user of a product from which the sample is obtained. The end-user may provide a sample or a portion of the sample to be analyzed (e.g., by mailing a single prescription pill to a service provider). In some instances, the sample may be received from the end-user at predetermined intervals. For example, the sample may be received every day, every 3 days, every week, bi-weekly, every month, every 3 months, every 6 months, every year, or less frequently from the end-user. In some instances, a single sample (e.g. a single pill) may be received from the end-user at a time (e.g. at the predetermined intervals). Alternatively, two, three, four, five, six, seven, eight, nine, ten, twenty, or more samples may be received from the end-user at a time (e.g. at the predetermined intervals). The samples may comprise a same ingredient (e.g. same API). Alternatively, differing samples with differing ingredients (e.g. differing APIs) may be received at a time.

An end-user may be desirous of verifying or confirming the identity of the sample. For example, an end-user may be prescribed a pharmaceutical sample (e.g., a medication) and may want to confirm the composition of the sample. For example, the end-user may wish to confirm that a specific active ingredient is present in the sample. Alternatively or in addition, the end-user may wish to identify an inactive ingredient, or multiple inactive ingredients in a sample. In other examples, the end-user may wish to confirm that a specific amount of an active ingredient is present in the sample. In yet other examples, the end-user may wish to confirm the purity of the sample (i.e., the absence of impurities in the sample). The sample may be tested utilizing the methods, devices, and systems as set forth herein to determine one or more characteristics of the sample. The one or more characteristics may include the identity of a molecule in the sample, the quantity of a molecule in the sample, or both. In some cases, the one or more characteristics may include a chemical identity (ID) of the sample, or a chemical of the sample. A chemical ID may refer to a precise chemical identity of the sample, or a chemical of the sample. In some instances, a chemical ID may refer to a unique signature of the chemical. Optionally, the chemical ID may refer to a particular molecular configuration. In some instances, the chemical ID may refer to a unique spectra of the sample (or a chemical of the sample). In some instances, no two chemicals may have a same signature (e.g. chemical ID). In some instances, a chemical ID may refer to a name in chemical nomenclature or a registry number assigned by an organization service, such as the chemical abstract service (CAS). In some instances, the chemical ID may provide insight into impurities existing in the sample.

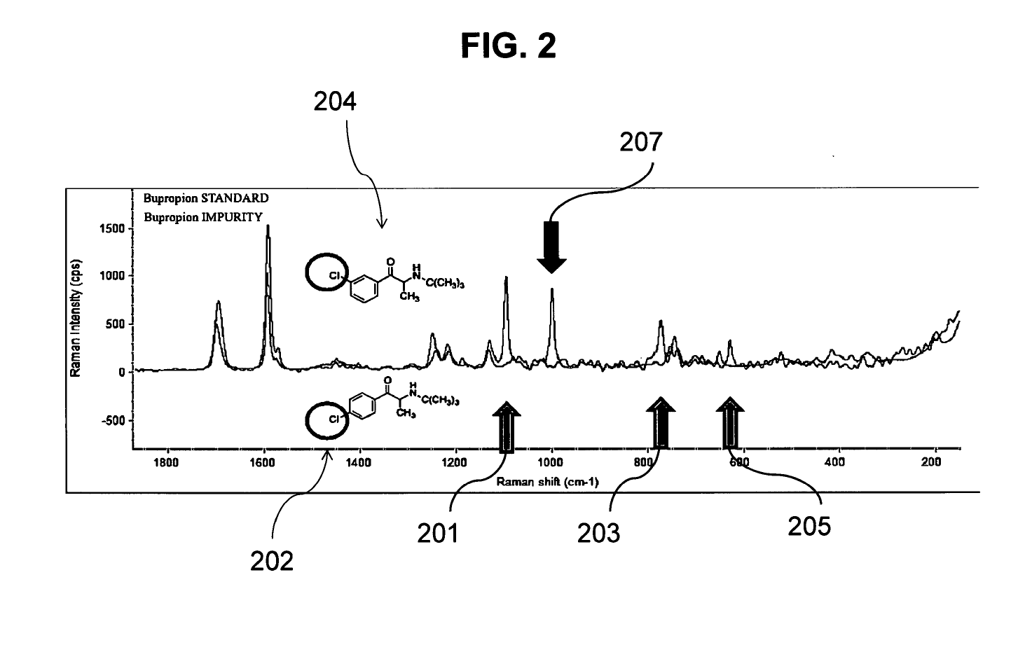

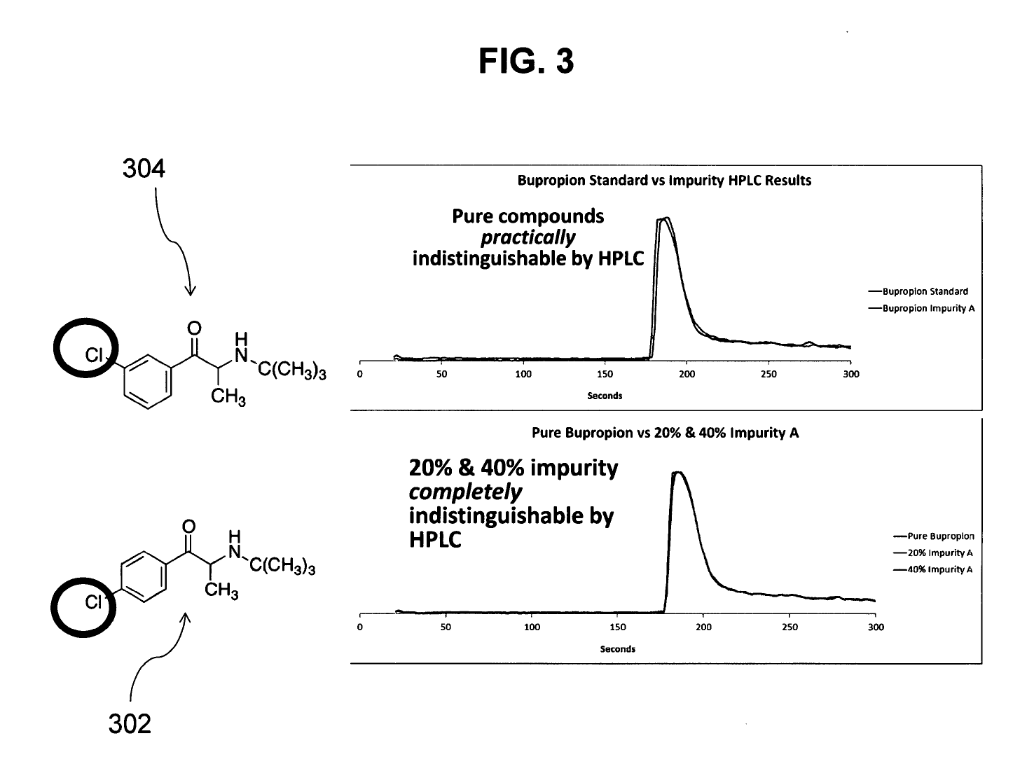

FIG. 2 depicts a non-limiting example of detection of impurities in a sample utilizing the methods and systems described herein. As shown in FIG. 2, an impurity 202 of bupropion was identified and distinguished from a bupropion standard 204 utilizing the methods, devices, and systems described herein. As an example, bupropion standard has a 3-chloro group whereas bupropion impurity has a 4-chloro group. As illustrated, a bupropion standard and bupropion impurity differ in peaks exhibited for certain Raman shift wavenumbers. For example, a Raman intensity for the bupropion impurity is higher and/or distinguishable as compared to a Raman intensity for the bupropion standard at Raman shifts 201, 203, 205. As another example, a Raman intensity for the bupropion standard is higher and/or distinguishable as compared to a Raman intensity for the bupropion impurity at Raman shift 207. The methods, devices, and systems were capable of identifying the 3-chloro to 4-chloro change, whereas alternative methods (e.g., high-performance liquid chromatography (HPLC)) may be unable to distinguish between the bupropion standard and a bupropion impurity. FIG. 3 depicts a system unable to detect impurities in a sample. As illustrated in FIG. 3, pure compounds 304 are practically indistinguishable by HPLC from impure compounds 302. FIGS. 2 and 3 may illustrate that no two chemicals have a same chemical ID (e.g. signature, unique spectra, etc) in Raman whereas two chemicals with the same molecular weight and same empirical formula may have the same signature in HPLC, as evidenced by the bupropion and its impurity.

In some aspects, the one or more characteristics may include the identity and/or quantity of more than one ingredient in the sample. For example, the methods may identify 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 different ingredients in the sample. In some cases, the methods may identify one or more characteristics of the more than one ingredient substantially simultaneously. In some aspects, the one or more characteristics may include the identity and/or quantity of more than one excipient in the sample. For example, the methods may identify 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 different excipients in the sample. In some cases, the methods may identify one or more characteristics of the more than one excipient substantially simultaneously.

The methods described herein may be particularly suited to analyzing samples of varying shapes and/or sizes. Optionally, the methods described herein may be particularly suited to analyzing samples of varying densities. In some instances, the methods described herein may be particularly suited to analyzing samples received from differing manufacturers of the sample. For example, the methods may be suitable for analyzing the composition of different samples containing a same ingredient but of differing size and shape without a calibration step prior to the analysis. For example, the methods may be suitable for analyzing the composition of different samples containing a same ingredient but of differing size and shape without a calibration step between analyzing a first sample and analyzing a second sample. For example, the methods may be capable of identifying the API for a plurality of different samples without separate calibration. For example, the methods may be capable of identifying a quantity of an API for a plurality of different samples without separate calibration. In this instance, the plurality of different samples may comprise different pills. In some cases, the plurality of different samples may comprise different shapes and different sizes. In some cases, the plurality of different samples may be dissolved or suspended in a solvent prior to the analysis. The sample(s), substantially as described throughout, may comprise an ingredient of interest and may be in any given form, e.g. solid, liquid, gelatinous, etc.

In some aspects, the methods involve preparing a liquid sample for analysis. In particular cases, the methods involve dissolving a sample to generate a solution. In one example, the sample may be provided in a solid form (e.g., a pill or tablet). The solid sample can be dissolved in a liquid to generate a solution for analysis. Dissolving the sample may involve crushing or pulverizing the sample prior to the addition of one or more solvents. For example, a pill or tablet may be crushed with a steel ball in a tube prior to addition of a solvent. In other examples, the sample may be crushed in the presence of a solvent. In some instances, the liquid sample comprise an extract of the sample. In other cases, the liquid sample may be a liquid solution. In other cases, the liquid sample may be a liquid suspension. In some instances, the liquid sample may be further processed prior to analysis. For example, the liquid sample may be placed in a filter such as a spin filter to get a final extract utilized in the analysis.

In particular aspects, the methods provide for the use of a solvent. The solvent may be used to dissolve a sample to generate a solution for analysis. In some instances, enough solvent may be added to ensure an ingredient (e.g. API) of the sample is dissolved. In some instances, enough solvent may be added to ensure all of the ingredient (e.g. API) of the sample is dissolved. In some instances, the ingredient may be distributed in the solution. In some instances, the ingredient may be distributed homogeneously within the solution. Accordingly, a subsample (e.g. component, part, etc) of the solution may be representative of the whole. The use of solvents and/or the crushing may make a particular size and/or shape of the sample irrelevant for purposes of analysis.

In some cases, the choice of solvent may provide improvements to traditional analytical methods. Non-limiting examples of solvents that may be amenable to performing the methods described herein include: pentane; cyclopentane; hexane; cyclohexane; benzene; toluene; 1,4-dioxane; chloroform; diethyl ether; dichloromethane (DCM), tetrahydrofuran (THF); ethyl acetate; acetone; dimethylformamide (DMF); acetonitrile (MeCN); dimethyl sulfoxide (DMSO); DMSO/DMF; nitromethane; propylene carbonate; formic acid; n-butanol; isopropanol (IPA); n-propanol; ethanol; methanol; acetic acid; water; acetaldehyde; 1,2-butanediol; 1,3-butanediol; 1,4-butanediol; 2-butoxyethanol; butyric acid; diethanolamine; diethylenetriamine; dimethoxyethane; ethylamine; ethylene glycol; furfuryl alcohol; glycerol; methyl diethanolamine; methyl isocyanide; 1-propanol; 1,3-propanediol; 1,5-pentanediol; 2-propanol; propanoic acid; propylene glycol; pyridine; triethylene glycol; 1,2-dimethylhydrazine; unsymmetrical dimethylhydrazine; hydrazine; hydrofluoric acid; hydrogen peroxide; nitric acid; sulfuric acid; 1-butanol; 2-butanol; 2-butanone; t-butyl alcohol; carbon tetrachloride; chlorobenzene; 1,2-dichloroethane; diethylene glycol; bis(2-methoxyethyl)ether (diglyme); 1,2-dimethoxy-ethane (glyme, DME); heptane; hexamethylphosphoramide (HMPA); hexamethylphosphorous triamide (HMPT); methyl t-butyl ether (MTBE); methylene chloride; N-methyl-2-pyrrolidinone (NMP); nitromethane; petroleum ether (ligroine); triethyl amine; o-xylene; m-xylene; p-xylene; 1-chlorobutane; N,N-diisopropylethylamine; Cap B (80% tetrahydrofuran, 10% pyridine, 10% 1-methylimidazole); Cap Mix A (90% tetrahydrofuran, 10% acetic anhydride); Cap Mix A (80% tetrahydrofuran, 10% acetic anhydride, 10% 2,6-lutidine); Cap Mix A (80% tetrahydrofuran, 10% acetic anhydride, 10% pyridine); trifluoroacetic acid; 1,1,1-trichloroethane; 1,2-dichloroethane; 1-octanol; 2,2,4-trimethylpentane; 2-butanone; 2-methoxyethanol; 2-methyl-1-propanol; 3-methyl-1-butanol; 4-methyl-2-pentanone; benzyl alcohol; butyl acetate; carbon disulfide; carbon tetrachloride; chlorobenzene; dichloromethane; diisopropyl ether; formamide; nitrobenzene; nitromethane; tert-butanol; tetrachloroethylene; trichloroethylene; 1,1,2,2-Tetrachloroethane; 1,2,3,4-Tetrahydronaphthalene reagent grade, 1-hexanol; 2-butoxyethyl acetate; 2-methoxyethyl acetate; 2-pentanone; 3-pentanone; cyclopentane; decahydronaphthalene; diethylene glycol diethyl ether; ethylene glycol diethyl ether; isopentyl acetate; methyl acetate; methyl formate; nitromethane; propionaldehyde; tert-butyl acetoacetate; trichloroethylene; triethyl orthoformate; 1,2,4-trichlorobenzene; 1,2-dichlorobenzene; 1,2-dichloroethane; 1,2-dimethoxyethane; 1,3-dioxolane; 1,4-dioxane; 1-chlorobutane; 1-methoxy-2-propanol; 2-(2-butoxyethoxy) ethyl acetate; 2,2,4-trimethylpentane; 2-butanone; 2-butoxyethanol; 2-ethoxyethanol; 2-ethylhexyl acetate; 2-heptanone; 2-methoxyethanol; 2-methyl-1-propanol; 2-methylbutane; 2-methyltetrahydrofuran; 3-methyl-1-butanol; 4-]methyl-2-pentanone; 5-methyl-2-hexanone; anisole; benzonitrile; decane; dibutyl ether; formaldehyde diethyl acetal; diethylene glycol butyl ether; diethylene glycol monoethyl ether; diethylene glycol monoethyl ether acetate; dodecane; ethyl 3-ethoxypropionate; ethylbenzene; 2-propoxyethanol; hexadecane; isopropyl acetate; methyl cyclohexane; N,N-dimethylacetamide; nonane; propyl acetate; TEBOL 99; or tetrahydropyran.

In some instances, water may be added to the solvent to prevent absorption from the air and improve the precision of the analysis. Alternatively or in addition, water may be added to lower a vapor pressure of the solvents. For example, water may be added to the solvent at about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.2%, 1.4%, 1.6%, 1.8%, 2%, 2.5%, 3%, 3.5%, 4%, 4.5%, 5%, 7%, 10%, or more than 10%.

In some cases, the solvent is non-volatile. Volatility may refer to a tendency of a substance to vaporize and may be directly related to the vapor pressure of the solvent. At a given temperature, a solvent with a higher vapor pressure vaporizes more readily than a substance with a lower vapor pressure. In some cases, the solvent has a vapor pressure of less than 40 mm Hg at 20° C. Non-limiting examples of solvents with a vapor pressure of less than 40 mm Hg at 20° C. include acetic acid; acetyl acetone; 2-aminoethanol; aniline; anisole; benzonitrile; benzyl alcohol; 1-butanol; 2-butanol; 1-butanol; 2-butanone; chlorobenzene; cyclohexanol; cyclohexanone; diethylene glycol; dimethylformamide (DMF); dimethylsulfoxide (DMSO); ethyl acetoacetate; ethylene glycol; 1-hexanol; 1-pentanol; 2-pentanol; 3-pentanol; 1-propanol; pyridine; toluene; 1-Methyl-2-pyrrolidinone; propylene carbonate; 2-Pyrrolidinone; Pyrrolidinone; 1,3-Butanediol; water; p-xylene; or any combination of the aforementioned solvents with other solvents or solutions. In some cases, the solvent has a vapor pressure of less than 100, 90, 80, 70, 60, 50, 40, 30, 20, 10, or 5 mm Hg at 20° C.

In some aspects, the solvent is selected to have one or more of the following properties: (1) low volatility; (2) high drug solubility; (3) low excipient solubility; (4) low toxicity; and (5) a favorable spectra which enables precise analysis. In some cases, the solvent is selected such that the one or more ingredients are dissolved in the solvent but the one or more excipients are not dissolved in the solvent. In some cases, the undissolved excipients can be filtered or otherwise removed from the sample.

In some aspects, the solvent is selected based on the one or more ingredients present in the sample. The solvent may be selected to have high drug solubility. In some cases, the solvent has a drug solubility of at least about 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 100 mg/mL, 200 mg/mL, 300 mg/mL, 400 mg/mL, 500 mg/mL, 600 mg/mL, 700 mg/mL, 800 mg/mL, 900 mg/mL, 1000 mg/mL or greater.

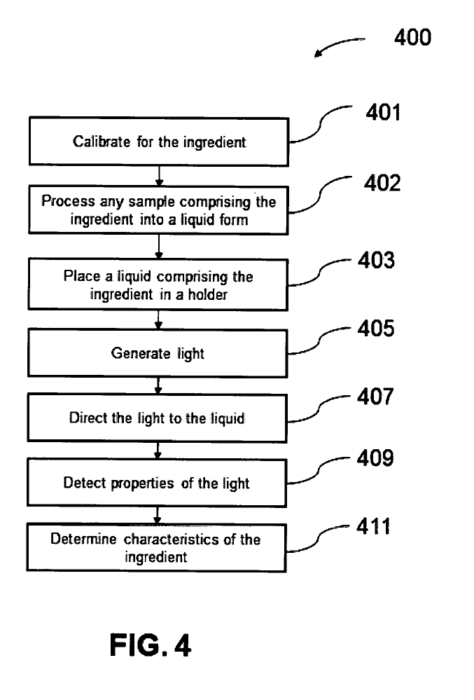

In some aspects, the methods involve the use of a device and/or system for analysis of the solution (e.g. liquid). FIG. 4 describes a method 400 for determining characteristics of an ingredient in a sample. As one example, the characteristics may be an amount (e.g. mass) of the ingredient in the sample. While determining an amount of the ingredient is primarily discussed herein, it is to be understood that any other characteristic (e.g. chemical identity, presence, etc) of the ingredient may be determined using the method described herein. In some instances, the method 400 may optionally comprise a step 402 of processing the sample into a liquid form. For example, the sample may be a solid sample such as a pill or tablet. In some instances, the sample may be crushed or pulverized. In some instances, the crushed or pulverized sample may be mixed with one or more solvents. In some instances, the sample may be crushed in the presence of a solvent. As previously described herein, any processing the sample into a liquid for may enable the method to be utilized for any sample comprising the ingredient, e.g. without calibrating specifically for features of the sample.

In some instances, an amount (e.g. mass) of solvent that is added may be measured and/or recorded. The amount of the ingredient in the sample may be determined by finding a ratio of the ingredient (e.g. dissolved ingredient) to the solvent. The methods, devices, and systems provided herein may be utilized to analyze some of the liquid (e.g. sample in liquid form) and determine the relative ratio of dissolved ingredient to solvent in it.

In some instances, to determine the ratio (e.g., the relative concentration of the API), a basis for comparison may be necessary and/or helpful. Accordingly, the method may comprise a step 401 of calibrating for the ingredient. The ingredient may be substantially as described above. For example, the ingredient may be an ingredient within the sample such as an API. In some instances the step of calibrating is accomplished using reference standards. A reference standard may be a standardized substance which is used as a measurement base for same or similar substances. A reference standard may be utilized for creating reference standard concentrations. In some instances, the reference standard concentrations may be linearly independent.

Optionally, the step of calibrating for the ingredient may be accomplished using about three, four, five, six, seven, eight, nine, ten, fifteen, twenty, twenty-five, thirty, fifty, one-hundred, two-hundred, or more reference standard concentrations. In some instances, the step of calibrating for the ingredient may be accomplished using four or less reference standard concentrations. In some instances, using a larger number of reference-standard concentrations may minimize measurement errors and/or noise. In some instances, the step of calibrating for the ingredient may be accomplished in equal to or less than about 24 hours, 18 hours, 12 hours, 9 hours, 6 hours, 3 hours, 2 hours, 1 hours, or 30 minutes. The step of calibrating the system for the ingredient may render the systems provided herein to be utilized universally for samples of different sizes, shapes, and compositions, e.g. without intermediary calibration steps. The step of calibrating the system for the ingredient may render the systems provided herein to be utilized universally for samples of different sizes, shapes, and compositions comprising the ingredient, e.g. without intermediary calibration steps. In some instances, the methods, devices, and systems may be configured to analyze a plurality of liquid samples obtained from different samples (e.g. pills) without further calibration steps. In some instances, the methods, devices, and systems may be configured to analyze a plurality of liquid samples obtained from different pills having the same ingredient without further calibration steps.

In some instances, the calibration step may comprise calibrating for a given ingredient and solvent pair. In some instances the calibrating step may comprise taking a spectra of the reference standards. The reference standards may refer to solutions of the ingredient (e.g. API) in the solvent, for which the relative concentration of the ingredient is known precisely. In some instances, a number of reference standards with differing concentrations may be made. For example, one, two, three, four, five, six, seven, eight, nine, ten, fifteen, twenty, twenty-five, thirty, fifty, one-hundred, two-hundred, or more reference standard concentrations may be made. A spectrum of each of the different reference standard concentrations may be measured and/or collected. Subsequently, quantification algorithms can use those data (e.g. collected spectra) as the requisite bases for comparison. Optionally, a spectrum from a sample solution with an unknown concentration of the ingredient can be input and a relative concentration of the ingredient to solvent may be computed utilizing the bases as a comparison to determine characteristics of the ingredient in the sample.

Few reference standard concentrations may be utilized while still enabling the systems and methods to perform substantially as described throughout. In some instances, reference standard concentrations equal in number to about or fewer than 3, 4, 5, 6, 7, 8, 9, 10, 20, 40, or 100 may be utilized while still enabling the systems and methods to perform substantially as described throughout. In some instances, the need for few reference standard concentrations may enable the calibration technique to be carried out much faster than traditionally possible while still enabling the systems and methods to perform substantially as described throughout. In some instances, the need for few reference standard concentrations may enable the calibrating step to be accomplished in equal to or less than about 24 hours, 18 hours, 12 hours, 9 hours, 6 hours, 3 hours, 2 hours, 1 hours, or 30 minutes.

As described throughout, the calibrating step may be performed once for a given ingredient (e.g. API) and it may not be necessary to perform a new calibration process for different samples that contain the same ingredient. For example a sample may comprise a same ingredient but may comprise differing compositions, sizes, weights, shapes, excipients, etc. Nevertheless the devices and systems provided herein may be universally applicable for the different samples (e.g. processed samples) after the initial calibration step that calibrates for the given ingredient. As an example, a calibration that is performed for acetaminophen in a given solvent may be applied to oblong 500 mg acetaminophen pills from manufacturer A, round 100 mg acetaminophen pills from manufacturer B, and so on. As another example, a calibration that is performed for acetaminophen in a given solvent may be applied to oblong 500 mg acetaminophen pills from manufacturer A, round 100 mg acetaminophen pills from manufacturer B, and so on, even if the inactive ingredients in those pills are different.

In some instances, the calibrating step may comprise selecting pairs of locations (i.e., wave-numbers) at which the ratio of spectral intensities is compared. While a single pair may be utilized for the calibrating step, a plurality of different pairs may be utilized in some instances. In some instances, using a plurality of different pairs may enable a more accurate and robust determination of the characteristics of the ingredients. In some instances, the pairs of locations may be tailored so as to avoid regions where there may be interference from other ingredients (e.g. dissolved excipients) that may interfere with determination of characteristics of the ingredient. In some instances, the optimal pairs of locations may be selected autonomously, with the aid of one or more processors.

In some instances, the calibrating step may utilize a database. In some instances, the database may comprise information regarding a known ingredient (e.g. API) for a particular sample and excipients within the particular sample. In some instances, the information from the database may be utilized to determine possible or relevant excipients that may be encountered when analyzing a sample (e.g. pill) containing a given ingredient (e.g. API). In some instances, a spectra of excipients dissolved in the solvents may be measured or recorded, and spectral features of the excipients' spectra may be determined. Subsequently, when selecting for the optimal spectral location-pairs to use in the calibration, those locations that lie in regions where relevant excipients are found to have spectral activity may be avoided in being selected.

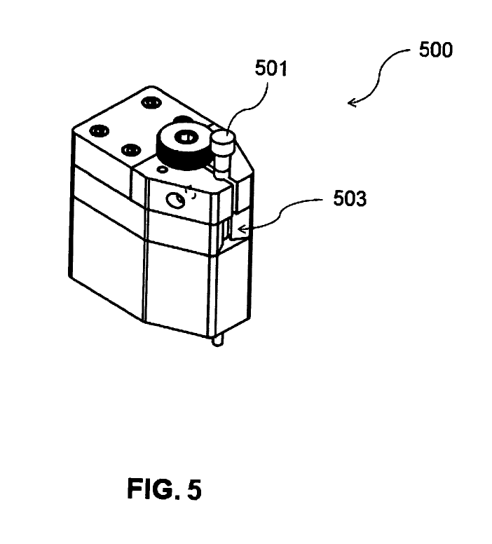

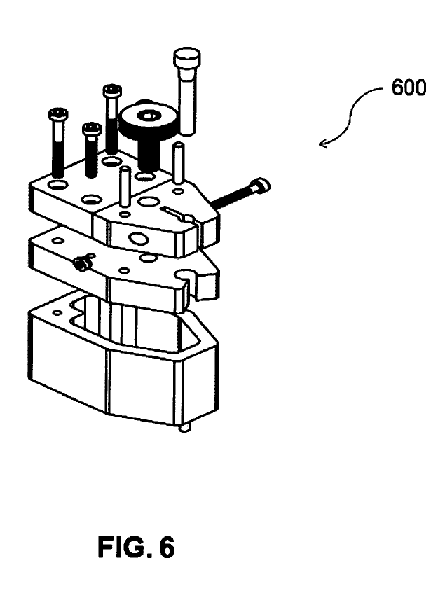

The methods may comprise a step 403 of placing the solution into a holder of a device. The holder may be a vial or vessel that is capable of receiving a liquid sample. Alternatively or in addition, the holder may be configured to receive a solid sample. The liquid sample or solution may be as described herein and may comprise the sample and/or a solvent. In some instances, the liquid sample may comprise an ingredient of interest (e.g. API) and a solvent. Optionally, the liquid sample may comprise excipients and/or water. In some cases, the holder of the device is secured to the device by a mechanism. In some cases, the mechanism is a clamp. The clamp may be of any configuration that secures the vial holder to the device. In some instances, the mechanism may ensure that the holder is substantially in a same position for implementing analysis of a liquid sample or solution. In some instances, the mechanism may ensure that the holder is substantially in a same position for implementing analysis of a plurality of liquid samples or solutions. The plurality of liquid samples or solutions may comprise samples and/or ingredients from a plurality of different samples or products. In some instances, the mechanism may ensure consistency of a background fluorescence level. For example, the mechanism may ensure that a background fluorescence level is consistent across a plurality of experiments or measurements concerning a same or different samples such that the background fluorescence may be easily subtracted and/or discounted as being contributing factor to any subsequent measurements or determinations. A non-limiting example of a clamp that is suitable for use with the methods, devices, and systems described herein is shown in FIGS. 5 and 6.

In some cases, the methods may further comprise a step 405 of generating light with the aid of an optical source. In some cases, the light is a monochromatic light. In some instances, the light is a light beam. Optionally, the light is a laser beam. The light can be in the ultraviolet range (e.g., about 10 nm to about 390 nm), the visible range (e.g., about 390 nm to about 700 nm) or the near infrared range (e.g., from about 700 nm to about 2500 nm).

In some cases, the method may further comprise a step 407 of directing, with aid of an optical arrangement, the light to the solution. The light directed to the liquid may interact with the solution. In some cases, the interaction comprises light being reflected or scattered off of the solution. In other cases, the interaction comprises light being transmitted through the solution.

In some cases, the methods may further comprise step 409 of detecting, with aid of a detector, properties of the light subsequent to the interaction. In some cases, the light is directed to a first side of the sample and scattered light is detected at the first side. Optionally, an inelastic scattering of the light may be detected by the detector. In other cases, the light is directed to a first side of the sample and transmitted light is detected at the other side of the sample. In some cases, the device is a Raman spectrometer. In this example, a monochromatic light (e.g., a laser) is directed at the solution-based sample. The photons of the laser light are absorbed by the sample and then reemitted. The frequency of the reemitted photons is shifted up or down. This shift in frequency can be detected and may provide information regarding the composition of the sample. In some cases, the device is configured to perform Raman spectroscopy on the sample. In some cases, the device is configured to perform back-scattering Raman spectroscopy on the sample. In other cases, the device is configured to perform transmission Raman spectroscopy on the sample.

In some aspects, the methods described herein may comprise a step to remove or filter out background noise. In some instances, the background noise may be a regular ambient background baseline. The regular ambient background baseline may be a background noise existing as a part of the system. In some instances, the ambient background baseline may be a constant. The ambient background baseline (e.g. constant) may be subtracted off. Alternatively or in addition, the background noise may be due to a fluorescence of the holder. For example, fluorescence of the vial in which the liquid sample (e.g. solution) is contained in while implementing the method may contribute to a background noise. In one example, a spectral scan is obtained of an empty vial at exactly the same position where a spectral scan of the full vial (i.e., with sample) is to be subsequently taken. The empty vial spectra may then be subtracted from the full vial spectra, thus eliminating or reducing background fluorescence. In some cases, the clamp mechanism may secure the vial to the device such that multiple measurements at the same position of the vial can be made. For example, the clamp may secure the vial to the device such that the vial is unable to move during the addition or removal of sample. Optionally, the background noise may be due to a fluorescence of a liquid (e.g. solvent, solution, etc) contained in the holder. In some instances, the background noise due to a fluorescence of the liquid may be mitigated or eliminated via low-frequency filters and/or low-order polynomial corrections or projections.

In some cases, the method may further comprise a step 411 of determining characteristics of the ingredient. The determining may be based on the detected properties of the light. In addition, the determining may be based on the information gathered from the calibration step. In some instances, the characteristics of the ingredient may comprise an identity of the ingredient. Alternatively or in addition, the characteristics of the ingredient may comprise a quantity of the ingredient. In some instances, the quantity of the ingredient may be determined based on a relative concentration of the ingredient to the solvent. For example, the quantity of the ingredient may be estimated based on a relative concentration of the ingredient to the solvent. In some instances, estimating the quantity of the ingredient based on a relative concentration of the ingredient to the solvent may be useful to account for other substances (e.g. excipients) which may be included in the liquid. In some instances, the quantity of the ingredient may be measured (e.g. determined, quantified, estimated) with an error equal to or less than 3%. In some instances, the characteristics of the ingredient may comprise an impurity of the ingredient. Optionally, the characteristics of the ingredient may comprise information regarding a chemical ID of the ingredient. For example, step 411 may provide information regarding existence of impurities within the sample, substantially as described with respect to FIG. 2. As an example, a spectra of an ingredient may be known. The sample spectrum that is observed (e.g. via method 400) may be compared to the known spectra for a given ingredient to check for a chemical ID.

In some instances, the amount, or quantity of the ingredient (e.g. API content) may further be characterized. Accordingly, the characteristics of the sample may comprise a standard error for the ingredient of interest, or bootstrap confidence interval for the amount of the ingredient. For example, an uncertainty, or standard error for the amount may be determined. In some instances, a bootstrap distribution for the amount of the ingredient may be generated for the sample and/or an estimation of uncertainty for individual samples may be determined. The standard error, uncertainty of the quantification of the amount of the ingredient, and/or bootstrap confidence interval for the amount may be generated or provided for each individual sample. For example, utilizing the ratiometric approach, e.g. described with respect to the calibration step, a plurality of possible estimates for the amount of the ingredient may be determined in order to determine a possible distribution of the amount. For example, the amount of ingredient in a sample may be estimated using a plurality of different ratios, and a bootstrap distribution and/or confidence interval for the amount of ingredient in the sample may be generated.

In some instances, the method may determine characteristics of a second ingredient in the sample substantially simultaneously with the ingredient. Optionally, the method may determine characteristics of a third, fourth, fifth, sixth, seventh, eighth, ninth, or tenth ingredient in the sample substantially simultaneously with the ingredient.

In some aspects, the methods may involve removing the sample and the holder from the mechanism (e.g. clamp) after analysis. The mechanism may be prepared to receive a second sample. In some instances, the mechanism may be prepared to receive a second sample in a second holder. A second sample may be added and analysis may be performed on the second sample. In some cases, the second sample may be analyzed without the need for an additional calibration step. In some cases, the first and the second sample may be different (i.e., of a different composition). In some cases, the second sample may be analyzed with the same or similar performance characteristics as the first sample (e.g., high accuracy, high precision, rapid sample-to-analysis time, etc.). The method described herein may take place in a setting that is not environmentally controlled. For example, it may be unnecessary to control for temperature, humidity, air pressure, microbial content, and/or contaminant content while performing method 400. In some instances, none of the steps of the method may take place in an environmentally controlled setting. Optionally, some of the steps may take place in an environmentally controlled setting while other steps may take place outside the environmentally controlled setting. Lack of need for an environmentally controlled setting may contribute to simplicity and/or ease of use of the present methods, devices, and systems. In some instances, the method described herein may use chemicals and/or procedures that do not require protective equipment. For example, by utilizing non-volatile and/or non-hazardous materials (e.g. solvents) in implementing the method 400, no protective equipment may be necessary, contributing to simplicity and/or ease of use of the present methods, devices, and systems. In some instances, all, or parts of method 400 may be substantially automated. For example steps 402 through 411 may be substantially automated that a user or operator may only need to input the sample (e.g. solid sample). Optionally, some of the steps may be substantially automated. For example, processing of the sample (e.g. crushing, adding solvent, etc) may be accomplished by a user or operator while other steps of the method may be substantially automated without further operator input. Partial or whole automation of the method may contribute to simplicity and/or ease of use of the present methods, devices, and systems. In some instances, the method may be accomplished with minimal supervision and/or with minimal special technical training, e.g. based at least in part on the aforementioned simplicity and or ease of use.

In some instances, the method 400 may take place within a small area. For example, the entirety of the method 400 may be undertaken in an area equal to or less than about 1 m², 5000 cm², 4000 cm², 3000 cm², 2500 cm², 2000 cm², 1500 cm², 1000 cm², 800 cm², 600 cm², 400 cm², 300 cm², 200 cm², or 100 cm². In some instances, the method 400 may take place with aid of a self-contained, or integrated device. For example, a device may be configured to perform the method 400. Optionally, the device may be configured to perform parts of the method 400. In some instances, the device may comprise a maximum dimension equal to or less than about 100 cm, 80 cm, 60 cm, 50 cm, 45 cm, 40 cm, 35 cm, 30 cm, 25 cm, 20 cm, 15 cm, or 10 cm. In some instances, the method may take place in a device comprising a volume equal to or less than about 1 m³, 500000 cm³, 250000 cm³, 100000 cm³, 50000 cm³, 25000 cm³, 20000 cm³, 15000 cm³, 10000 cm³, 8000 cm³, 6000 cm³, 5000 cm³, 4000 cm³, 3000 cm³, 2000 cm³, or 1000 cm³.

Optionally, the one or more characteristics of the sample may be presented on a report and provided to the end-user. In some cases, the report may present the composition of the sample. For example, the report may present the identity of one or more molecules present in the sample, the quantity of one or more molecules present in the sample, or both. In some cases, the report may indicate the presence of an active ingredient present in the sample and additionally, the presence of one or more excipients, one or more impurities, and the like. In some cases, the report may provide a list of all of the ingredients found within a sample and/or their quantities. In some cases, the report may confirm that the sample is of the intended composition (e.g., is authentic). In other cases, the report may confirm that the sample is of an unintended composition (e.g., counterfeit). The report may be presented in any number of different ways, including alphanumerical presentation, graphical presentation and the like. In some cases, the report may include spectral data represented graphically. In some cases, the report may summarize the results of the analysis in any number of lists, tables, charts, and the like. The report may be presented to an end-user in a tangible form (e.g., on a sheet of paper) or may be presented to in an electronic format. Electronic reports may be accessed via, e.g., the Internet or by e-mail. In some cases, the report is presented for display on a screen. In some cases, the report is uploaded to a database. Optionally, information contained in the database may be viewed or downloaded by a user. The database may comprise a compilation of reports containing information regarding the composition of a plurality of different samples obtained from various sources. The database in some instances may provide utility in identifying sources of counterfeit or improperly formulated pharmaceutical products.

Systems

Disclosed herein are systems for performing the methods described herein. The systems may include any number of devices that operate individually or in concert to perform the described methods. The minimum components of the system are described herein, however, it shall be understood that additional components may be used. Although examples are herein described, it is to be further understood that any order or arrangement of the system components may be utilized.

In some aspects, a system is provided for determining characteristics of an ingredient in a sample. The system comprises: a) a calibration means for calibrating the system for the ingredient; b) a holder for holding a liquid comprising the ingredient; c) an optical source configured to generate light; d) an optical arrangement configured to direct the generated light to the liquid such that the light interacts with the sample; e) a detector configured to detect properties of the light subsequent to said interaction; and f) one or more processors, individually or collectively, configured to determine the characteristics of the ingredient. Optionally, the sample may be processed into a liquid form, e.g. to make the liquid comprising the ingredient that is placed in the holder.

In other aspects, a system is provided for determining characteristics of a sample comprising an active pharmaceutical ingredient (API). The system comprises: a) a holder for holding a solution, wherein the solution comprises the sample and a solvent; b) an optical source configured to generate light; c) an optical arrangement configured to direct the generated light to the solution such that the light interacts with the sample; d) a detector configured to detect properties of the light subsequent to said interaction; and e) one or more processors, individually or collectively, configured to determine the characteristics of the sample, wherein the characteristics comprise an API content of the sample, and wherein the system determines the API content with less than 3% error. Optionally, the sample may be processed into a liquid form, e.g. to make the liquid comprising the ingredient that is placed in the holder. Optionally, the system may comprise a calibration means for calibrating the system for the ingredient.

In other aspects, a system is provided for determining characteristics of a sample comprising an active pharmaceutical ingredient (API). The system comprises: a) a holder for holding a solution, wherein the solution comprises the sample and a solvent; b) an optical source configured to generate light; c) an optical arrangement configured to direct the generated light to the solution such that the light interacts with the sample; d) a detector configured to detect properties of the light subsequent to said interaction; and e) one or more processors, individually or collectively, configured to determine the characteristics of the sample, wherein the characteristics comprise an API content of the sample, and wherein the system determines the characteristics of the sample within 30 minutes beginning with generating the light. Optionally, the sample may be processed into a liquid form, e.g. to make the liquid comprising the ingredient that is placed in the holder. Optionally, the system may comprise a calibration means for calibrating the system for the ingredient.

In particular aspects, the system may comprise one or more devices. As described above, in some instances, a device (e.g. self-contained device) may be provided for accomplishing the method 400. Alternatively or in addition, a plurality of devices may be utilized in concert to accomplish the method 400. In some instances, the one or more devices comprise an optical source. In some cases, the optical source provides light to the system. The light may be a monochromatic light. In some instances, the light is a light beam. Optionally, the light is a laser beam. The light can be in the ultraviolet range (e.g., about 10 nm to about 390 nm), the visible range (e.g., about 390 nm to about 700 nm) or the near infrared range (e.g., from about 700 nm to about 2500 nm). The system may further comprise one or more optical arrangements. The one or more optical arrangements may direct the light generated by the optical source to a sample. Any number of optical arrangements may be utilized and may include, for example, the use of one or more filters, lenses, mirrors, prisms, beam splitters, diffraction gratings, etc. The system may further include a wavelength selector such as a filter or a spectrophotometer. The wavelength selector may restrict the wavelength of light being transmitted to the sample. The system may further include one or more detectors. The one or more detectors may be configured to detect light from the system. In one example, the system is configured such that the optical source provides light to the sample and the detector is configured to receive and detect scattered or reflected light from the sample. In another example, the system is configured such that the optical source provides light to the sample and the detector is configured to receive and detect light transmitted through the sample. Non-limiting examples of detectors that may be used with the system include photodiode arrays, charge-coupled devices (CCDs) or photo-multiplier tubes (PMTs). In some cases, the system includes a Raman spectrometer. The Raman spectrometer may be a back-scattering Raman spectrometer. In other cases, the Raman spectrometer may be a transmission Raman spectrometer.

In certain aspects, the system may comprise a holder. In some instances, the device may be configured to receive a holder such as a vessel or vial. In some cases, the holder contains a sample. In some instances, the holder contains an ingredient (e.g. API) from the sample. The holder may additionally comprise liquid or a solvent. In some instances, the holder may contain a solution or a suspension. In some cases, the holder is empty (e.g., for background measurements). The device may position the holder such that a portion of the holder (and the sample therein) can be illuminated by a light source.

In some cases, the system may include a mechanism for securing the holder to the device. FIG. 5 depicts a perspective view of a non-limiting mechanism 500 for securing the holder 501 to the device. In some cases, the mechanism is a clamp. The mechanism may be used to secure the vial or vessel to the holder such that the vial or vessel is unable to move during the addition or removal of the sample or reagents. This may allow for multiple measurements to be taken at the exact same position of the sample. In some instances, the mechanism may clamp the holder in a same position such that the spectra of the holder may be scanned before adding a liquid sample (e.g. solution comprising the ingredient) and after adding the liquid sample. Afterwards, by subtracting spectra of the empty holder at the particular point, background spectra (e.g. from an empty holder) may be eliminated since the holder is scanned at an exact same position or location. In some instances, the gasket may be configured to clean the holder between analyzing different liquid samples. In some instances, the mechanism may comprise an opening 503 for allowing the light (e.g. generated from the light source) to interact with, or scan, the vial and/or liquid sample. FIG. 6 depicts an exploded view of a non-limiting mechanism 600 for securing the holder to the device.

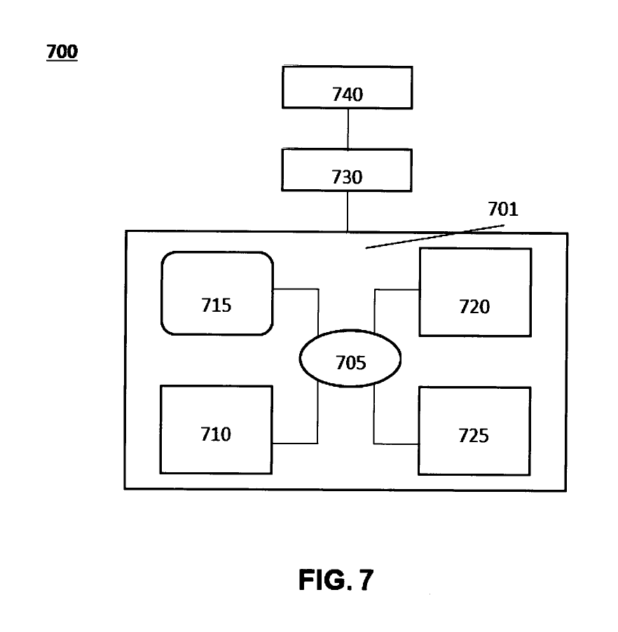

The devices or systems described may be operably coupled to one or more computer systems. FIG. 7 shows a computer system 701 programmed or otherwise configured to implement the methods of the disclosure and to function with the devices and systems described herein, such as receiving spectral data, analyzing the spectral data generating a report. The computer system 701 includes a central processing unit (CPU, also “processor” and “computer processor” herein) 705, which can be a single core or multi core processor, or a plurality of processors for parallel processing. The computer system 701 also includes memory 710 (e.g., random-access memory, read-only memory, flash memory), electronic storage unit 715 (e.g., hard disk), communications interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communications bus (solid lines), such as a motherboard. The storage unit 715 can be a data storage unit (or data repository) for storing data. The computer system 701 is operatively coupled to a computer network (“network”) 730 with the aid of the communications interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730 in some cases, with the aid of the computer system 701, can implement a peer-to-peer network, which may enable devices coupled to the computer system 701 to behave as a client or a server.

The computer system 701 is in communication with a processing system 735. The processing system 735 can be configured to implement the methods disclosed herein, such as determining one or more characteristics of the sample, generating a report, and uploading the report to a database. The processing system 735 can be in communication with the computer system 701 through the network 730, or by direct (e.g., wired, wireless) connection. The processing system 735 can be configured for analysis, such as analyzing spectral data.

Methods and systems as described herein can be implemented by way of machine (or computer processor) executable code (or software) stored on an electronic storage location of the computer system 701, such as, for example, on the memory 710 or electronic storage unit 715. During use, the code can be executed by the processor 705. In some examples, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, can be compiled during runtime or can be interpreted during runtime. The code can be supplied in a programming language that can be selected to enable the code to execute in a pre-compiled, as-compiled or interpreted fashion.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display that comprises a user interface (UI) for providing, for example, a report to an end-user. Examples of UI's include, without limitation, a graphical user interface (GUI) and web-based user interface.

In some embodiments, the computer system 701 includes a display to provide visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein. The display may provide one or more biomedical reports to an end-user as generated by the methods described herein.

In some embodiments, the computer system 701 includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

The computer system 701 can include or be operably coupled to one or more databases. The databases may comprise genomic, proteomic, pharmacogenomic, biomedical, pharmaceutical, chemical, and/or scientific databases. The databases may be publicly available databases. Alternatively, or additionally, the databases may comprise proprietary databases. The databases may be commercially available databases. In some cases, spectral data generated by the methods and devices provided herein may be uploaded to the database.

Data can be produced and/or transmitted in a geographic location that comprises the same country as the user of the data. Data can be, for example, produced and/or transmitted from a geographic location in one country and a user of the data can be present in a different country. In some cases, the data accessed by a system of the disclosure can be transmitted from one of a plurality of geographic locations to a user. Data can be transmitted back and forth among a plurality of geographic locations, for example, by a network, a secure network, an insecure network, an internet, or an intranet.

Performance

In some aspects, the methods devices, and systems provide improvements to existing technologies. These improvements may include, without limitation, universal application to pharmaceutical end-products, faster sample-to-analysis time, improved accuracy, improved precision, and decreased costs.

In some cases, the methods, devices, and systems provide for rapid sample processing time. For example, the sample processing time to generate a sample solution may be on the order of less than about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In some cases, the sample analysis time (e.g., Raman collection time) can be on the order of less than about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes. In some cases, the sample-to-analysis time (e.g., from the beginning of sample processing time to the end of the Raman collection time) can be on the order of less than about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes.

In some cases, the methods, devices, and systems provide for improved accuracy. Accuracy is a measurement of how closely a value conforms to the correct value. In some cases, the methods, devices, and systems analyze the quantity of an ingredient in the sample with an accuracy of about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0% or greater than 10.0%.

In some cases, the methods, devices, and systems determine the amount of an ingredient (e.g., an API) in the sample with an error rate equal to or less than about 10%, 9.9%, 9.8%, 9.7%, 9.6%, 9.5%, 9.4% 9.3%, 9.2%, 9.1%, 9.0%, 8.9%, 8.8%, 8.7%, 8.6%, 8.5%, 8.4%, 8.3%, 8.2%, 8.1%, 8.0%, 7.9%, 7.8%, 7.7%, 7.6%, 7.5%, 7.4%, 7.3%, 7.2%, 7.1%, 7.0%, 6.9%, 6.8%, 6.7%, 6.6%, 6.5%, 6.4%, 6.3%, 6.2%, 6.1%, 6.0%, 5.9%, 5.8%, 5.7%, 5.6%, 5.5%, 5.4%, 5.3%, 5.2%, 5.1%, 5.0%, 4.9%, 4.8%, 4.7%, 4.6%, 4.5%, 4.4%, 4.3%, 4.2%, 4.1%, 4.0%, 3.9%, 3.8%, 3.7%, 3.6%, 3.5%, 3.4%, 3.3%, 3.2%, 3.1%, 3.0%, 2.9%, 2.8%, 2.7%, 2.6%, 2.5%, 2.4%, 2.3%, 2.2%, 2.1%, 2.0%, 1.9%, 1.8%, 1.7%, 1.6%, 1.5%, 1.4%, 1.3%, 1.2%, 1.1%, 1.0%, 0.9%, 0.8%, 0.7%, 0.6%, 0.5%, 0.4%, 0.3%, 0.2%, 0.1% or less.

In some cases, the methods, devices, and systems provide for improved precision. Precision may refer to the closeness of two or more measurements and may reflect the amount of variability in the system. In some cases, the methods, devices, and systems analyze the quantity of an ingredient in the sample with a precision equal to or less than about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, about 5.0%, about 5.1%, about 5.2%, about 5.3%, about 5.4%, about 5.5%, about 5.6%, about 5.7%, about 5.8%, about 5.9%, about 6.0%, about 6.1%, about 6.2%, about 6.3%, about 6.4%, about 6.5%, about 6.6%, about 6.7%, about 6.8%, about 6.9%, about 7.0%, about 7.1%, about 7.2%, about 7.3%, about 7.4%, about 7.5%, about 7.6%, about 7.7%, about 7.8%, about 7.9%, about 8.0%, about 8.1%, about 8.2%, about 8.3%, about 8.4%, about 8.5%, about 8.6%, about 8.7%, about 8.8%, about 8.9%, about 9.0%, about 9.1%, about 9.2%, about 9.3%, about 9.4%, about 9.5%, about 9.6%, about 9.7%, about 9.8%, about 9.9%, about 10.0% or greater than 10.0%.

In some aspects, the methods, devices, and systems may improve the ability to detect an impurity in a sample. In some aspects, the methods, devices, and systems may improve the ability to distinguish between an impurity and an active ingredient in a sample. In some cases, the methods and devices may be capable of detecting an impurity in a sample containing the impurity and an active ingredient. In some cases, the methods and devices are capable of detecting an impurity in a sample that contains about 0.05%, 0.1%, 0.15%, 0.2%, 0.25%, 0.3%, 0.35%, 0.4%, 0.45%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or greater than 99% of the impurity.

### Particular Embodiments

The following examples are given for the purpose of merely providing particular embodiments of the invention and are not meant to limit the present invention in any fashion.

In one aspect, a method of determining one or more characteristics of an ingredient in a sample using Raman Spectroscopy is provided. The method comprises: a) calibrating for the ingredient; b) processing the sample into a liquid comprising the ingredient; c) placing the liquid comprising the ingredient into a holder; d) generating light with aid of an optical source; e) directing, with aid of an optical arrangement, the light to the liquid; f) detecting, with aid of a detector, inelastic scattering of the light subsequent to an interaction between the light and the liquid; and g) determining, with aid of one or more processors individually or collective, the one or more characteristics of the ingredient comprising a quantity of the ingredient in the sample.

In some embodiments, the one or more characteristics of the ingredient comprise an identity of the ingredient. Optionally, the quantity of the ingredient may be measured with an error equal to or less than 3%. The one or more characteristics of the sample may additionally comprise a standard error for the quantity of the ingredient, a distribution of the quantity of the ingredient or both. In some instances, the amount, or quantity of the ingredient may further be characterized. For example, an uncertainty, or standard error for the amount may be determined. In some instances, a bootstrap distribution for the amount of the ingredient may be generated. In some embodiments, the one or more characteristics of the ingredient comprise an impurity of the ingredient. Alternatively or in addition, the one or more characteristics of the ingredient may comprise information regarding a chemical ID of the ingredient. Optionally, the ingredient has a molecular weight of 1000 Daltons or less. In some embodiments, the ingredient is an active pharmaceutical ingredient (API). Alternatively or in addition, the ingredient is a peptide. Alternatively or in addition, the ingredient is a protein. In some embodiments, the ingredient is a small molecule. In some embodiments, the calibrating is accomplished using 10 reference standards or less. In some embodiments, the calibrating may render the method able to be utilized universally for samples of different sizes, shapes, formulations, or physical forms. In some embodiments, the liquid is a liquid solution. Alternatively, the liquid may be a liquid suspension. In some embodiments, the interaction comprises light being transmitted through the liquid. Alternatively or in addition, the interaction comprises light being reflected from the liquid. In some embodiments, the method is implemented using a Raman spectroscopy device. In some embodiments, the method determines characteristics of one or more other ingredients in the sample substantially simultaneously with the ingredient. In some embodiments, the method comprises an initial step of receiving the sample from an individual end-user of the sample. In some embodiments, the method further comprises generating a report for the individual end-user. Optionally, the method may further comprise uploading the report to a database. In some embodiments, the liquid comprises a solvent. Optionally, the solvent is non-volatile. Furthermore, the solvent may comprise a vapor pressure of 40 mm Hg or less at 20° C. In some embodiments, the method further comprises securely holding the holder with a mechanism. In some embodiments, the mechanism is a clamp. In some embodiments, the method further comprises measuring a background fluorescence of the holder prior to placing the liquid in the holder. In some embodiments, the method further comprises filtering the sample prior to the placing to remove one or more excipients therefrom. In some embodiments, steps b)-g) are accomplished in equal to or less than 30 minutes. Optionally, steps b)-g) are accomplished in equal to or less than 15 minutes.

In another aspect, a Raman Spectroscopic system is provided. The system may be capable of performing any of the aforementioned methods and may comprise: a) a calibration mechanism for calibrating the system for the ingredient; b) a processing means for processing the sample into a liquid comprising the ingredient; c) a holder for holding the liquid comprising the ingredient; d) an optical source configured to generate light; e) an optical arrangement configured to direct the generated light to the liquid; f) a detector configured to detect inelastic scattering of the light subsequent to an interaction between the light and the liquid; and g) one or more processors, individually or collectively, configured to determine the one or more characteristics of the ingredient.

In some embodiments, the system further comprises a mechanism for securely holding the holder. In some embodiments, the mechanism comprises a clamp.

In another aspect, a method of generating a report on one or more characteristics of a sample for an individual is provided. The method comprises: a) obtaining the sample, wherein the sample comprises an active pharmaceutical ingredient (API); b) determining, with aid of one or more devices, the one or more characteristics of the sample; and c) generating, with aid of one or more processors operably coupled to the one or more devices, a report for the individual, wherein the report comprises the one or more characteristics of the sample comprising an API content of the sample, and wherein the individual is an end-user of the sample.

In some embodiments, the method further comprises, prior to the determining, preparing, with aid of a solvent, the sample for analysis. In some embodiments, the solvent is non-volatile. In some embodiments, the solvent comprises a vapor pressure of 40 mm Hg or less at 20° C. In some embodiments, the one or more characteristics of the sample comprises information regarding a chemical ID of the sample. In some embodiments, the API content is determined with an error equal to or less than 5%. In some embodiments, the API content is determined with an error equal to or less than 3%. The one or more characteristics of the sample may additionally comprise a standard error for the API content, an API distribution of the sample, or both. In some instances, the amount, or quantity of the ingredient (e.g. API content) may further be characterized. For example, an uncertainty, or standard error for the amount may be determined. In some instances, a bootstrap distribution for the amount of the ingredient may be generated. In some embodiments, the one or more devices comprise a Raman spectroscopy device. In some embodiments, the one or more devices comprise a holder configured to hold the sample. In some embodiments, the holder is secured to the one or more devices via a mechanism. In some embodiments, the mechanism is a clamp. In some embodiments, a fluorescence of the holder is measured in the absence of the sample to determine a background fluorescence prior to the determining. In some embodiments, the one or more devices comprise a gasket configured to clean the holder. In some embodiments, the one or more devices is configured to determine one or more characteristics of an API for a plurality of different samples without further calibration. In some embodiments, the plurality of different samples are different medications, or pharmaceutical formulations. In some embodiments, the plurality of different samples are of different shapes and/or sizes. In some embodiments, the method further comprises recording the one or more characteristics of the sample to a database. In some embodiments, the method further comprises repeating the steps of obtaining, determining, generating, and recording for a plurality of different samples comprising the API. In some embodiments, the method further comprises presenting an aggregate result for the plurality of different samples. In some embodiments, steps b) and c) are accomplished in equal to or less than 30 minutes. In some embodiments, the sample is a single pill. In some embodiments, the API has a molecular weight of 1000 Daltons or less. In some embodiments, the API is a protein. In some embodiments, the API is a small molecule. In some embodiments, the API is a peptide. In some embodiments, the device comprises a filter for removing one or more excipients from the sample.

In another aspect, a method for determining one or more characteristics of a sample comprising an active pharmaceutical ingredient (API) is provided. The method comprises: a) placing a solution in a holder, wherein the solution comprises the sample and a solvent; b) generating light with aid of an optical source; c) directing, with aid of an optical arrangement, the light to the sample; d) detecting, with aid of a detector, one or more properties of the light subsequent to an interaction between the light and the solution; and e) determining, with aid of one or more processors individually or collectively, the one or more characteristics of the sample, wherein the one or more characteristics comprise an API content of the sample, and wherein the API content is determined with less than 3% error.

The one or more characteristics of the sample may additionally comprise a standard error for the API content, an API distribution of the sample, or both. In some instances, the amount, or quantity of the ingredient (e.g. API content) may further be characterized. For example, an uncertainty, or standard error for the amount may be determined. In some instances, a bootstrap distribution for the amount of the ingredient may be generated. In some embodiments, the method further comprises processing the sample comprising the API into the solution prior to the step of placing the solution in the holder. In some embodiments, processing the sample comprises crushing the sample and adding the solvent. In some embodiments, the one or more processors are further configured to generate a report on the API content of the sample. In some embodiments, the one or more processors are further configured to upload the report to a database. In some embodiments, the one or more characteristics comprise a chemical ID of the sample. In some embodiments, the method is capable of determining the API for a plurality of different samples without further calibration. In some embodiments, the plurality of different samples comprise different medications. In some embodiments, the plurality of different samples are of different shapes, sizes, formulations, or physical forms. In some embodiments, the interaction of light with the solution results in inelastic scattering of the light. In some embodiments, the method further comprises securely holding the holder with a mechanism. In some embodiments, the mechanism is a clamp. In some embodiments, the method further comprises measuring a background fluorescence of the holder prior to the placing. In some embodiments, the solvent is non-volatile. In some embodiments, the solvent comprises a vapor pressure of 40 mm Hg or less at 20° C. In some embodiments, the sample is a medication received from an end-user of the medication. In some embodiments, the method further comprises filtering the sample prior to the placing to remove one or more excipients from the sample.

In another aspect, a system for determining one or more characteristics of a sample comprising an active pharmaceutical ingredient (API) is provided. The system may be capable of performing any of the abovementioned methods and may comprise: a) a holder for holding a solution, wherein the solution comprises the sample and a solvent; b) an optical source configured to generate light; c) an optical arrangement configured to direct the generated light to the solution; d) a detector configured to detect one or more properties of the light subsequent to an interaction between the light and the solution; and e) one or more processors, individually or collectively configured to determine the one or more characteristics of the sample, wherein the one or more characteristics comprise an API content of the sample, and wherein the system determines the API content of the sample with less than 3% error.

The one or more characteristics of the sample may additionally comprise a standard error for the API content, an API distribution of the sample, or both. In some instances, the amount, or quantity of the ingredient (e.g. API content) may further be characterized. For example, an uncertainty, or standard error for the amount may be determined. In some instances, a bootstrap distribution for the amount of the ingredient may be generated. In some embodiments, the system further comprises a mechanism for securely holding the holder. In some embodiments, the mechanism is a clamp.

In another aspect, a method for determining one or more characteristics of a sample comprising an active pharmaceutical ingredient (API) is provided. The method comprises: a) placing a solution in a holder, wherein the solution comprises the sample and a solvent; b) generating light with aid of an optical source; c) directing, with aid of an optical arrangement, the light to the sample; d) detecting, with aid of a detector, properties of the light subsequent to an interaction between the light and the solution; and e) determining, with aid of one or more processors individually or collectively, the one or more characteristics of the sample, wherein the one or more characteristics comprise a API content of the sample, and wherein b) through e) take place within 30 minutes.

In some embodiments, the one or more processors are further configured to generate a report on the API content of the sample. In some embodiments, the one or more processors are further configured to upload the report to a database. In some embodiments, the one or more characteristics comprise a chemical ID of the sample. In some embodiments, the method is configured to determine the API for a plurality of different samples without calibration. In some embodiments, the plurality of different samples are different medications. In some embodiments, the plurality of different samples are of different shapes, sizes, formulations, or physical forms. In some embodiments, the interaction of light with the solution results in inelastic scattering of the light. In some embodiments, the method further comprises securely holding the holder with a mechanism. In some embodiments, the mechanism is a clamp. In some embodiments, the method further comprises measuring a background fluorescence of the holder prior to placement of the solution in the holder. In some embodiments, the solvent is non-volatile. In some embodiments, the solvent comprises a vapor pressure of 40 mm Hg or less at 20° C. In some embodiments, the sample is a medication received from an end-user of the medication. In some embodiments, the method further comprises filtering the sample prior to the placing to remove one or more excipients from the sample.

In another aspect, a system for determining one or more characteristics of a sample comprising an active pharmaceutical ingredient (API) is provided. The system may be capable of performing any of the abovementioned methods and may comprise: a) a holder for holding a solution, wherein the solution comprises the sample and a solvent; b) an optical source configured to generate light; c) an optical arrangement configured to direct the generated light to the solution; d) a detector configured to detect properties of the light subsequent to an interaction between the light and the solution; and e) one or more processors, individually or collectively configured to determine the one or more characteristics of the sample, wherein the one or more characteristics comprise an API content of the sample, and wherein the system determines the one or more characteristics of the sample within 30 minutes.

In some embodiments, the system further comprises a mechanism for securely holding the holder. In some embodiments, the mechanism comprises a clamp.

In another aspect, a method of determining one or more characteristics of an active pharmaceutical ingredient (API) in a sample using Raman Spectroscopy is provided. The method comprises: a) calibrating for the API; b) processing the sample into a liquid comprising the API and a solvent; c) placing the liquid into a holder; d) generating light with aid of an optical source; e) directing, with aid of an optical arrangement, the light to the liquid; f) detecting, with aid of a detector, inelastic scattering of the light subsequent to an interaction between the light and the liquid; and g) determining, with aid of one or more processors individually or collective, a concentration of the API in the solvent.

In some embodiments, the concentration is determined through a ratiometric analysis of the API and the solvent. In some embodiments, two or more analyte concentrations are determined simultaneously through a ratiometric analysis of analytes and solvent. In some embodiments, spectral features of the API and solvent are chosen for ratiometric calibration with the aid of a database of known interfering substances, so as to minimize possible interference and optimize accuracy. In some embodiments, the API and the solvent are calibrated through a ratiometric approach utilizing 10 or less spectral features on a spectrum. In some embodiments, the API and the solvent are calibrated by utilizing 5 or less spectral features on a spectrum. In some embodiments, the API and the solvent are calibrated through a ratiometric approach utilizing 2 features of a spectrum. Optionally, the API and the solvent are calibrated through a ratiometric approach utilizing one or more combinations of spectral features. In some embodiments, the API and the solvent are calibrated through a ratiometric approach utilizing 100 or more combinations of spectral features. In some embodiments, the API and the solvent are calibrated through a ratiometric approach utilizing 1,000 or more combinations of spectral features. In some embodiments, the API and the solvent are calibrated through a ratiometric approach utilizing 10,000 or more combinations of spectral features. In some embodiments, API and the solvent are calibrated through a ratiometric approach utilizing 100,000 or more combinations of spectral features. In some embodiments, the API and the solvent are calibrated through a ratiometric approach utilizing 1,000,000 or more combinations of spectral features. Optionally, the API and the solvent are calibrated through a ratiometric approach utilizing 10,000,000 or more combinations of spectral features. In some embodiments, an automated optimization of analytical calibration for specific analytes is used. In some embodiments, an automated optimization of analytical calibration for specific solvents is used. In some embodiments, an automated optimization of analytical calibration for selecting calibration-relevant spectral features is used. In some instances, an estimation of uncertainty of the measurement may be reportable for each, individual sample uniquely.

### EXAMPLES

The following examples are given for the purpose of merely providing examples the invention and are not meant to limit the present invention in any fashion. The present examples, along with the methods described herein are exemplary, and are not intended as limitations on the scope of the invention. Changes therein and other uses which are encompassed within the spirit of the invention as defined by the scope of the claims will occur to those skilled in the art.

### Example 1

### Identification of the Composition of a Pharmaceutical Sample

An end-user purchases a drug at a convenience store or over the Internet. The end-user is suspicious of the contents of the drug and mails a single pill to a service provider. The service provider receives the single pill for analysis. Regardless of the composition, dosage or source of manufacture of the pill, the service provider crushes the pill to form a powder and dissolves the pill in a solvent that is selected to be low toxicity, high drug solubility, low volatility, low excipient solubility, and/or generates a spectra favorable for analysis. An empty vial is placed into a holder of a device and clamped to the holder. Raman spectroscopy is performed on the empty vial to generate a background spectrum. The sample is added to the vial and Raman spectroscopy is performed on the full vial at the exact position as was performed on the empty vial to generate a test spectrum. The background spectrum is subtracted from the test spectrum. An active pharmaceutical ingredient (API) is identified and quantified in the sample with an accuracy of better than 3%. An impurity is further detected in the sample. The result of the analysis is presented on a report and sent via email to the end-user.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Additionally, any details described with respect to specific embodiments may be applicable to any other embodiments described throughout. It is intended that the following claims define the scope of the invention and that methods and structures within the scope of these claims and their equivalents be covered thereby.

What is claimed is:

1. A method of determining one or more characteristics of an active pharmaceutical ingredient (API) in a sample using spectroscopy, the method comprising:
a) calibrating for the API;
b) processing the sample into a liquid comprising the API;
c) placing the liquid comprising the API into a holder;
d) generating light with aid of an optical source;
e) directing, with aid of an optical arrangement, the light to the liquid;
f) detecting, with aid of a detector, inelastic scattering of the light subsequent to an interaction between the light and the liquid; and
g) determining, with aid of one or more processors individually or collective, the one or more characteristics of the API comprising a quantity of the API in the sample.

2. The method of any of the preceding claims, wherein the method comprises an initial step of receiving the sample from an individual end-user of the sample.

3. The method of any of the preceding claims, further comprising generating a report for the individual end-user.

4. The method of any of the preceding claims, wherein calibrating for the API enables the method to be used universally for samples of different sizes, shapes, formulations, or physical forms.

5. The method of any of the preceding claims, wherein the method is performed using dispersive Raman spectroscopy.

6. The method of any of the preceding claims, wherein the one or more characteristics of the API comprises an impurity of the ingredient.

7. The method of any of the preceding claims, wherein the calibrating for the API step is accomplished using 20 reference standards or less.

8. The method of any of the preceding claims, wherein the 20 reference standards comprise solutions having the API in a solvent for which a relative concentration of the API is known precisely.

9. The method of any of the preceding claims, wherein the liquid is a liquid solution.

10. The method of any of the preceding claims, wherein the liquid is a liquid suspension.

11. The method of any of the preceding claims, wherein the interaction between the light and the liquid uses backscatter geometry.

12. The method of any of the preceding claims, wherein the liquid comprises a solvent.

13. The method of any of the preceding claims, wherein the solvent is non-volatile.

14. The method of any of the preceding claims, wherein the solvent comprises a vapor pressure of 40 mm Hg or less at 20° C.

15. The method of any of the preceding claims, further comprising filtering the sample prior to the step of placing the liquid into the holder.

16. The method of any of the preceding claims, wherein the step of calibrating for the API comprises calibrating for the API regardless of the sample's size, shape, formulation, or physical form such that steps b) through g) can be performed for samples of different sizes, shapes, formulations, or physical forms.

17. The method of any of the preceding claims, wherein the sample is a pharmaceutical formulation, and the step of calibrating for the API enables step b) through g) to be performed on any pharmaceutical formulation comprising the API.

18. A system for performing the method of any of the preceding claims, the system comprising:
a) a calibration mechanism for calibrating the system for the API;
b) a processing device for processing the sample into a liquid comprising the API;
c) a holder for holding the liquid comprising the API;
d) an optical source configured to generate light;
e) an optical arrangement configured to direct the generated light to the liquid;
f) a detector configured to detect inelastic scattering of the light subsequent to an interaction between the light and the liquid; and
g) one or more processors, individually or collectively, configured to determine the one or more characteristics of the API comprising a quantity of the API in the sample.

19. A method of generating a report on one or more characteristics of a sample for an individual, the method comprising:
a) obtaining the sample, wherein the sample comprises an active pharmaceutical ingredient (API);
b) determining, with aid of one or more devices, the one or more characteristics of the sample; and
c) generating, with aid of one or more processors operably coupled to the one or more devices, a report for the individual, wherein the report comprises the one or more characteristics of the sample comprising an API content of the sample, and wherein the individual is an end-user of the sample.

20. The method of any of the preceding claims, further comprising preparing the sample for analysis with aid of a solvent prior to the step of determining the one or more characteristics of the sample.

21. The method of any of the preceding claims, wherein the solvent is non-volatile.

22. The method of any of the preceding claims, wherein the solvent comprises a vapor pressure of 40 mm Hg or less at 20° C.

23. The method of any of the preceding claims, wherein the one or more devices comprises a backscatter Raman spectroscopy device.

24. The method of any of the preceding claims, wherein the one or more devices are configured to determine one or more characteristics of the API for a plurality of different samples without need to calibrate for each of the plurality of different samples.

25. The method of any of the preceding claims, wherein the plurality of different samples are of different shapes and sizes.

26. The method of any of the preceding claims, wherein the sample is a single pill.

27. The method of any of the preceding claims, wherein the one or more devices comprise a filter for removing one or more excipients from the sample.

28. The method of any of the preceding claims, further comprising a step of calibrating for the API regardless of the sample's size, shape, formulation, or physical form such that determining the one or more characteristics of the sample can be performed for samples of different sizes, shapes, formulations, or physical forms.

29. The method of any of the preceding claims, wherein the sample is a pharmaceutical formulation, and the step of calibrating for the API enables step b) to be performed on any pharmaceutical formulation comprising the API.

30. A method of determining one or more characteristics of an active pharmaceutical ingredient (API) in a sample using spectroscopy, the method comprising:
a) calibrating for the API;
b) processing the sample into a liquid comprising the API and a solvent;
c) placing the liquid into a holder;
d) generating light with aid of an optical source;
e) directing, with aid of an optical arrangement, the light to the liquid;
f) detecting, with aid of a detector, inelastic scattering of the light subsequent to an interaction between the light and the liquid; and
g) determining, with aid of one or more processors individually or collective, a concentration of the API in the solvent.

31. The method of any of the preceding claims, the concentration is determined through a ratiometric analysis of the API and the solvent.

32. The method of any of the preceding claims, two or more analyte concentrations are determined simultaneously through a ratiometric analysis of analytes and solvent.

33. The method of any of the preceding claims, wherein spectral features of the API and solvent are chosen for ratiometric calibration with the aid of a database of known interfering substances, so as to minimize possible interference and optimize accuracy.

34. The method of any of the preceding claims, wherein the API and the solvent are calibrated through a ratiometric approach utilizing 10 or less spectral features on a spectrum.

35. The method of any of the preceding claims, wherein the API and the solvent are calibrated by utilizing 5 or less spectral features on a spectrum.

36. The method of any of the preceding claims, wherein the API and the solvent are calibrated through a ratiometric approach utilizing 2 or more features on a spectrum.

37. The method of any of the preceding claims, wherein the API and the solvent are calibrated through a ratiometric approach utilizing one or more combinations of spectral features.

38. The method of any of the preceding claims, wherein the API and the solvent are calibrated through a ratiometric approach utilizing 100 or more combinations of spectral features.

39. The method of any of the preceding claims, wherein the API and the solvent are calibrated through a ratiometric approach utilizing 1,000 or more combinations of spectral features.

40. The method of any of the preceding claims, wherein the API and the solvent are calibrated through a ratiometric approach utilizing 10,000 or more combinations of spectral features.

41. The method of any of the preceding claims, wherein the API and the solvent are calibrated through a ratiometric approach utilizing 100,000 or more combinations of spectral features.

42. The method of any of the preceding claims, wherein the API and the solvent are calibrated through a ratiometric approach utilizing 1,000,000 or more combinations of spectral features.

43. The method of any of the preceding claims, wherein API and the solvent are calibrated through a ratiometric approach utilizing 10,000,000 or more combinations of spectral features.

44. The method of any of the preceding claims, wherein an automated optimization of analytical calibration for specific analytes is used.

45. The method of any of the preceding claims, wherein an automated optimization of analytical calibration for specific solvents is used.

46. The method of any of the preceding claims, wherein an automated optimization of analytical calibration for selecting calibration-relevant spectral features is used.

47. The method of any of the preceding claims, where an estimation of uncertainty of the measurement is reportable for each, individual sample uniquely.

48. The method of any of the preceding claims, wherein a non-parametric estimation of uncertainty of the measurement is reportable for each, individual sample uniquely.

49. The method of any of the preceding claims, wherein the method is performed using dispersive Raman spectroscopy.

50. The method of any of the preceding claims, wherein the one or more characteristics of the API comprises an impurity of the ingredient.

51. The method of any of the preceding claims, wherein the calibrating for the API step is accomplished using 20 reference standards or less.

52. The method of any of the preceding claims, wherein the 20 reference standards comprise solutions having the API in a solvent for which a relative concentration of the API is known precisely.

53. The method of any of the preceding claims, further comprising determining a quantity of the API in the sample.

54. The method of any of the preceding claims, wherein the step of calibrating for the API comprises calibrating for the API regardless of the sample's size, shape, formulation, or physical form such that steps b) through g) can be performed for samples of different sizes, shapes, formulations, or physical forms.

55. The method of any of the preceding claims, wherein the sample is a pharmaceutical formulation, and the step of calibrating for the API enables step b) through g) to be performed on any pharmaceutical formulation comprising the API.

56. The method of any of the preceding claims wherein the estimates are adjusted to correct for variation in the wavelength of the light directed to the liquid.

57. The method of any of the preceding claims wherein the calibration step includes calibrating for the adjustments used to correct for variation in the wavelength of the light directed to the liquid.

58. A system for performing the method of any of the preceding claims, the system comprising:
a) calibration mechanism for calibrating the system for the API;
b) a processing device for processing the sample into a liquid comprising the API and a solvent;
c) a holder for holding the liquid comprising the API and the solvent;
d) an optical source configured to generate light;
e) an optical arrangement configured to direct light to the liquid;
f) a detector configured to detect inelastic scattering of the light subsequent to an interaction between the light and the liquid; and
g) one or more processors, individually or collectively, configured to determine a concentration of the API in the solvent.
